(19) United States
(12) Patent Application Publication
(10) Pub. No.: US 2013/0039908 A1
(43) Pub. Date: Feb. 14, 2013

(54) PROGNOSTIC MARKERS AND METHODS FOR PROSTATE CANCER

(71) Applicants: Eddie Reed, Mobile, AL (US); Rui Gao, Bethesda, MD (US); William Douglas Figg, Fairfax, VA (US)

(72) Inventors: Eddie Reed, Mobile, AL (US); Rui Gao, Bethesda, MD (US); William Douglas Figg, Fairfax, VA (US)

(73) Assignee: University of South Alabama, Mobile, AL (US)

(21) Appl. No.: 13/641,066

(22) PCT Filed: Aug. 12, 2010
(86) PCT No.: PCT/US10/45383

§ 371 Date: Oct. 12, 2012

Related U.S. Application Data
(60) Provisional application No. 61/342,520, filed on Apr. 15, 2010.

(51) Int. Cl. C12Q 1/68; C40B 40/06; A61K 39/395; A61K 31/337; A61K 31/454; A61B 18/02; A61K 39/00; A61P 31/00; A61P 35/00; A61N 5/10; A61N 7/00; C40B 30/04; A61K 31/573

(52) U.S. Cl. 424/133.1; 435/6.11; 506/9; 506/16; 514/449; 514/323; 514/177; 424/277.1; 600/1; 601/2; 606/20

(57) ABSTRACT

The present invention relates to methods and compositions for the diagnosis, prognosis and treatment of neoplastic disorders. Some embodiments include methods, compositions, and kits for the prognosis and treatment of prostate cancer.

### CROSS-REFERENCE TO RELATED APPLICATIONS

This application claims priority to U.S. Provisional Application No. 61/342,520 filed on Apr. 15, 2010 entitled “GENETIC POLYMORPHISMS IN XRCC1 ASSOCIATED WITH RADIATION THERAPY IN PROSTATE CANCER”, the disclosure of which is hereby incorporated herein by reference in its entirety for any purpose.

### REFERENCE TO SEQUENCE LISTING

The present application is being filed along with a Sequence Listing in electronic format. The Sequence Listing is provided as a file entitled USA003SEQLIST.TXT, created Aug. 11, 2010, which is approximately 41 Kb in size. The information in the electronic format of the Sequence Listing is incorporated herein by reference in its entirety.

### FIELD OF THE INVENTION

The present invention relates to methods and compositions for the diagnosis, prognosis and treatment of neoplastic disorders. Some embodiments include methods and compositions for the prognosis and treatment of prostate cancer.

### BACKGROUND

Prostate cancer is the second most common cause of cancer related death and kills an estimated 37,000 people annually. The prostate gland, which is found exclusively in male mammals, produces several regulatory peptides. The prostate gland comprises stroma and epithelium cells, the latter group consisting of columnar secretory cells and basal non-secretory cells. A proliferation of these basal cells, as well as stroma cells gives rise to benign prostatic hyperplasia which is one common prostate disease. Another common prostate disease is prostatic adenocarcinoma, the most common of the fatal pathophysiological prostate cancers. Prostatic adenocarcinoma involves a malignant transformation of epithelial cells in the peripheral region of the prostate gland. Prostatic adenocarcinoma and benign prostatic hyperplasia are two common prostate diseases which have a high rate of incidence in the aging human male population. Approximately one out of every four males above the age of 55 suffers from a prostate disease of some form or another.

Prognosis in clinical cancer is an area of great concern and interest. It is important to know the aggressiveness of the malignant cells and the likelihood of tumor recurrence or spread in order to plan the most effective therapy. Prostate cancer, for example, is managed by several alternative strategies. In some cases local-regional therapy is utilized, consisting of surgery or radiation, while in other cases systemic therapy is instituted, such as chemotherapy or hormonal therapy.

Current treatment decisions for individual prostate cancer patients are frequently based on the stage of disease at diagnosis and the overall health or age of the patient. It has been reported that DNA ploidy can aid in predicting the course of disease in patients with advanced disease (Stage C and D1) (Lee et al., Journal of Urology 140:769-774 (1988)). In addition, the pretreatment level of the prostate specific antigen (PSA) has been used to estimate the risk of relapse after surgery and other types of treatment (Pisansky et al., Cancer 79:337-344 (1997)). However, a substantial proportion of patients with elevated or rising PSA levels after surgery remain clinically free of symptoms for extended periods of time (Frazier et al., Journal of Urology 149:516-518 (1993)). Therefore, even with these additional factors, practitioners are still unable to accurately predict the course of disease for all prostate cancer patients. The inability to differentiate tumors that will progress from those that will remain quiescent has created a dilemma for treatment decisions. There is clearly a need to identify new markers in order to separate patients with good prognosis who may not require further therapy from those more likely to relapse who might benefit from more intensive treatments.

Several side effects of surgical removal of the prostate gland (radical prostatectomy), radiation therapy and hormonal therapy have been documented. The side effects of surgery include discomfort with urination, urinary urgency, impotence, and the morbidity associated with general anesthesia and a major surgical procedure. Common complications associated with external-beam radiation therapy include impotence, discomfort with urination, urinary urgency, and diarrhea. The side effects of anti-androgen hormone therapy can include loss of libido, the development of breast tissue, and osteoporosis. Given the complications associated with some prostate cancer therapies, a marker that could distinguish between tumors that require aggressive treatments and those that require conservative treatment could result in higher survival rates and greater quality of life for prostate cancer patients. Thus, a need exists for a biomarker that can determine prostate cancer patient prognosis.

### SUMMARY

Some embodiments of the present invention include methods for evaluating a prognosis of a subject with a prostate neoplastic condition comprising: determining the genotype of said subject at at least one codon selected from the group consisting of the codon encoding amino acid 399 of the XRCC1 polypeptide, the codon encoding amino acid 194 of the XRCC1 polypeptide, and the codon encoding amino acid 762 of the PARP1 polypeptide.

In some embodiments, the step of determining the genotype comprises determining the identity of a polymorphic nucleotide selected from the group consisting of the polymorphic nucleotide at position 1316 of SEQ ID NO:17 or a polymorphic nucleotide corresponding thereto, the polymorphic nucleotide at position 700 of SEQ ID NO:17 or a polymorphic nucleotide corresponding thereto, and the polymorphic nucleotide at position 2456 of SEQ ID NO:19 or a polymorphic nucleotide corresponding thereto.

In some embodiments, the determining step the genotype comprises extending a primer that hybridizes to a sequence adjacent to the polymorphic nucleotide. In some embodiments, the determining the genotype comprises hybridizing a probe to a region that includes the polymorphic nucleotide.

Some embodiments also include obtaining a sample from said subject. In some embodiments, the sample comprises ex vivo genomic DNA.

Some embodiments also include providing the result of said determining step to a party in order for said party to select a treatment for said prostate neoplastic condition in said subject. In some embodiments, the party is a physician.

In some embodiments, the genotype is at least one genotype selected from the group consisting of XRCC1 R399Q AA, PARPI V762A CC, XRCC1 R194W CC, XRCC1 R399Q AG, XRCC1 R194W CT, and XRCC1 R399Q GG.

In some embodiments, the genotype is at least one genotype selected from the group consisting of AA for the polymorphic nucleotide at position 1316 of SEQ ID NO:17 or a polymorphic nucleotide corresponding thereto, CC for the polymorphic nucleotide at position 2456 of SEQ ID NO:19, CC for the polymorphic nucleotide at position 700 of SEQ ID NO:17 or a polymorphic nucleotide corresponding thereto, AG for the polymorphic nucleotide at position 1316 of SEQ ID NO:17 or a polymorphic nucleotide corresponding thereto, CT for the polymorphic nucleotide at position 700 of SEQ ID NO:17 or a polymorphic nucleotide corresponding thereto, and GG for the polymorphic nucleotide at position 1316 of SEQ ID NO:17 or a polymorphic nucleotide corresponding thereto.

In some embodiments, the presence of at least one genotype selected from the group consisting of XRCC1 R399Q AA, PARP1 V762A CC, and XRCC1 R194W CC indicates a favorable prognosis.

In some embodiments, the presence of at least one genotype selected from the group consisting of AA for the polymorphic nucleotide at position 1316 of SEQ ID NO:17 or a polymorphic nucleotide corresponding thereto, CC for the polymorphic nucleotide at position 2456 of SEQ ID NO:19 or a polymorphic nucleotide corresponding thereto, and CC for the polymorphic nucleotide at position 700 of SEQ ID NO:17 or a polymorphic nucleotide corresponding thereto indicates a favorable prognosis.

In some embodiments, the presence of CC for the polymorphic nucleotide at position 700 of SEQ ID NO:17 or a polymorphic nucleotide corresponding thereto and AA for the polymorphic nucleotide at position 1316 of SEQ ID NO:17 or a polymorphic nucleotide corresponding thereto together, or CC for the polymorphic nucleotide at position 700 of SEQ ID NO:17 or a polymorphic nucleotide corresponding thereto and AG for the polymorphic nucleotide at position 1316 of SEQ ID NO:17 or a polymorphic nucleotide corresponding thereto together indicates a favorable prognosis.

In some embodiments, the presence of at least one genotype selected from the group consisting of XRCC1 R194W CT and XRCC1 R399Q GG indicates an unfavorable prognosis.

In some embodiments, the presence of at least one genotype selected from the group consisting of CT for the polymorphic nucleotide at position 700 of SEQ ID NO:17 or a polymorphic nucleotide corresponding thereto and GG for the polymorphic nucleotide at position 1316 of SEQ ID NO:17 or a polymorphic nucleotide corresponding thereto indicates an unfavorable prognosis.

In some embodiments, the presence of XRCC1 R194W CT, and XRCC1 R399Q AG, or XRCC1 R399Q GG indicates an unfavorable prognosis.

In some embodiments, the presence of CT for the polymorphic nucleotide at position 700 of SEQ ID NO:17 or a polymorphic nucleotide corresponding thereto, and AG for the polymorphic nucleotide at position 1316 of SEQ ID NO:17 or a polymorphic nucleotide corresponding thereto, or GG for the polymorphic nucleotide at position 1316 of SEQ ID NO:17 or a polymorphic nucleotide corresponding thereto indicates an unfavorable prognosis.

In some embodiments, the prognosis comprises a favorable or unfavorable response to radiation therapy. In some embodiments, the prognosis comprises overall survival of said subject. In some embodiments, the favorable prognosis comprises a period for overall survival for said subject which is at least 1 year greater than the period of overall survival for a subject with an unfavorable prognosis. In some embodiments, the favorable prognosis comprises a period for overall survival for said subject which is at least 3 year greater than the period of overall survival for a subject with an unfavorable prognosis. In some embodiments, the favorable prognosis comprises a period for overall survival for said subject which is at least 6 year greater than the period of overall survival for a subject with an unfavorable prognosis.

Some embodiments also include administering a treatment for which the determined genotype is indicative of a favorable response. In some embodiments, the treatment is selected from surgery, radiation therapy, proton therapy, chemotherapy, cryosurgery, and high intensity focused ultrasound. In some embodiments, the radiation therapy is selected from external beam radiotherapy and brachytherapy.

In some embodiments, the condition is castrate-resistant prostate cancer.

In some embodiments, the subject is human.

In some embodiments, the determining is performed in an automated device.

Some embodiments of the present invention include methods for evaluating the response to radiation therapy in a subject with a prostate neoplastic condition comprising: determining the genotype of said subject at at least one codon selected from the group consisting of the codon encoding amino acid 399 of the XRCC1 polypeptide, the codon encoding amino acid 194 of the XRCC1 polypeptide, and the codon encoding amino acid 762 of the PARP1 polypeptide; and providing the result of said evaluating to a party in order for said party to select a treatment for said subject.

In some embodiments, the step of determining the genotype comprises determining the identity of a polymorphic nucleotide selected from the group consisting of the polymorphic nucleotide at position 1316 of SEQ ID NO:17 or a polymorphic nucleotide corresponding thereto, the polymorphic nucleotide at position 700 of SEQ ID NO:17, and the polymorphic nucleotide at position 2456 of SEQ ID NO:19 or a polymorphic nucleotide corresponding thereto. In some embodiments, the determining the genotype comprises extending a primer that hybridizes to a sequence adjacent to the polymorphic nucleotide. In some embodiments, the determining the genotype comprises hybridizing a probe to a region that includes the polymorphic nucleotide.

Some embodiments also include obtaining a sample from said subject. In some embodiments, the sample comprises ex vivo genomic DNA.

In some embodiments, the party is a physician.

In some embodiments, the genotype is at least one genotype selected from the group consisting of XRCC1 R399Q AA, PARPI V762A CC, XRCC1 R194W CC, XRCC1 R399Q AG, XRCC1 R194W CT, and XRCC1 R399Q GG.

In some embodiments, the genotype is at least one genotype selected from the group consisting of AA for the polymorphic nucleotide at position 1316 of SEQ ID NO:17 or a polymorphic nucleotide corresponding thereto, CC for the polymorphic nucleotide at position 2456 of SEQ ID NO:19, CC for the polymorphic nucleotide at position 700 of SEQ ID NO:17 or a polymorphic nucleotide corresponding thereto, AG for the polymorphic nucleotide at position 1316 of SEQ ID NO:17 or a polymorphic nucleotide corresponding thereto, CT for the polymorphic nucleotide at position 700 of SEQ ID NO:17 or a polymorphic nucleotide corresponding thereto, and GG for the polymorphic nucleotide at position 1316 of SEQ ID NO:17 or a polymorphic nucleotide corresponding thereto.

In some embodiments, the presence of at least one genotype selected from the group consisting of XRCC1 R399Q AA, PARPI V762A CC, and XRCC1 R194W CC indicates a favorable prognosis.

In some embodiments, the presence of at least one genotype selected from the group consisting of AA for the polymorphic nucleotide at position 1316 of SEQ ID NO:17 or a polymorphic nucleotide corresponding thereto, CC for the polymorphic nucleotide at position 2456 of SEQ ID NO:19 or a polymorphic nucleotide corresponding thereto, and CC for the polymorphic nucleotide at position 700 of SEQ ID NO:17 or a polymorphic nucleotide corresponding thereto indicates a favorable prognosis.

In some embodiments, the presence of CC for the polymorphic nucleotide at position 700 of SEQ ID NO:17 or a polymorphic nucleotide corresponding thereto and AA for the polymorphic nucleotide at position 1316 of SEQ ID NO:17 or a polymorphic nucleotide corresponding thereto together, or CC for the polymorphic nucleotide at position 700 of SEQ ID NO:17 or a polymorphic nucleotide corresponding thereto and AG for the polymorphic nucleotide at position 1316 of SEQ ID NO:17 or a polymorphic nucleotide corresponding thereto together indicates a favorable prognosis.

In some embodiments, the presence of at least one genotype selected from the group consisting of XRCC1 R194W CT and XRCC1 R399Q GG indicates an unfavorable prognosis.

In some embodiments, the presence of at least one genotype selected from the group consisting of CT for the polymorphic nucleotide at position 700 of SEQ ID NO:17 or a polymorphic nucleotide corresponding thereto and GG for the polymorphic nucleotide at position 1316 of SEQ ID NO:17 or a polymorphic nucleotide corresponding thereto indicates an unfavorable prognosis.

In some embodiments, the presence of XRCC1 R194W CT, and XRCC1 R399Q AG, or XRCC1 R399Q GG indicates an unfavorable prognosis.

In some embodiments, the presence of CT for the polymorphic nucleotide at position 700 of SEQ ID NO:17 or a polymorphic nucleotide corresponding thereto, and AG for the polymorphic nucleotide at position 1316 of SEQ ID NO:17 or a polymorphic nucleotide corresponding thereto, or GG for the polymorphic nucleotide at position 1316 of SEQ ID NO:17 or a polymorphic nucleotide corresponding thereto indicates an unfavorable prognosis

In some embodiments, the prognosis comprises overall survival of said subject. In some embodiments, the favorable prognosis comprises an overall survival at least 1 year greater than the overall survival of an unfavorable prognosis. In some embodiments, the favorable prognosis comprises an overall survival at least 3 years greater than the overall survival of an unfavorable prognosis. In some embodiments, the favorable prognosis comprises an overall survival at least 6 years greater than the overall survival of an unfavorable prognosis.

In some embodiments, the treatment is selected from surgery, radiation therapy, proton therapy, chemotherapy, cryosurgery, and high intensity focused ultrasound. In some embodiments, the radiation therapy is selected from external beam radiotherapy and brachytherapy.

In some embodiments, the condition is castrate-resistant prostate cancer.

In some embodiments, the subject is human.

In some embodiments, the determining is performed in an automated device.

Some embodiments of the present invention include methods for selecting a treatment for a subject with a prostate neoplastic condition comprising: determining the genotype of said subject at at least one codon selected from the group consisting of the codon encoding amino acid 399 of the XRCC1 polypeptide, the codon encoding amino acid 194 of the XRCC1 polypeptide, and the codon encoding amino acid 762 of the PARP1 polypeptide; and selecting a treatment for said subject based on the determined genotype.

In some embodiments, the step at determining the genotype comprises determining the identity of a polymorphic nucleotide selected from the group consisting the polymorphic nucleotide at position 1316 of SEQ ID NO:17 or a polymorphic nucleotide corresponding thereto, the polymorphic nucleotide at position 700 of SEQ ID NO:17 or a polymorphic nucleotide corresponding thereto, and the polymorphic nucleotide at position 2456 of SEQ ID NO:19 or a polymorphic nucleotide corresponding thereto.

In some embodiments, the determining the genotype comprises extending a primer that hybridizes to a sequence adjacent to the polymorphic nucleotide. In some embodiments, the determining the genotype comprises hybridizing a probe to a region that includes the polymorphic nucleotide.

Some embodiments also include obtaining a sample from said subject.

In some embodiments, the sample comprises ex vivo genomic DNA.

In some embodiments, the genotype is at least one genotype selected from the group consisting of XRCC1 R399Q AA, PARPI V762A CC, XRCC1 R194W CC, XRCC1 R399Q AG, XRCC1 R194W CT, and XRCC1 R399Q GG.

In some embodiments, the genotype is at least one genotype selected from the group consisting of AA for the polymorphic nucleotide at position 1316 of SEQ ID NO:17 or a polymorphic nucleotide corresponding thereto, CC for the polymorphic nucleotide at position 2456 of SEQ ID NO:19, CC for the polymorphic nucleotide at position 700 of SEQ ID NO:17 or a polymorphic nucleotide corresponding thereto, AG for the polymorphic nucleotide at position 1316 of SEQ ID NO:17 or a polymorphic nucleotide corresponding thereto, CT for the polymorphic nucleotide at position 700 of SEQ ID NO:17 or a polymorphic nucleotide corresponding thereto, and GG for the polymorphic nucleotide at position 1316 of SEQ ID NO:17 or a polymorphic nucleotide corresponding thereto.

In some embodiments, the presence of at least one genotype selected from the group consisting of XRCC1 R399Q AA, PARP1 V762A CC, and XRCC1 R194W CC indicates a favorable prognosis.

In some embodiments, the presence of at least one genotype selected from the group consisting of AA for the polymorphic nucleotide at position 1316 of SEQ ID NO:17 or a polymorphic nucleotide corresponding thereto, CC for the polymorphic nucleotide at position 2456 of SEQ ID NO:19 or a polymorphic nucleotide corresponding thereto, and CC for the polymorphic nucleotide at position 700 of SEQ ID NO:17 or a polymorphic nucleotide corresponding thereto indicates a favorable prognosis.

In some embodiments, the presence of CC for the polymorphic nucleotide at position 700 of SEQ ID NO:17 or a polymorphic nucleotide corresponding thereto and AA for the polymorphic nucleotide at position 1316 of SEQ ID NO:17 or a polymorphic nucleotide corresponding thereto together, or CC for the polymorphic nucleotide at position 700 of SEQ ID NO:17 or a polymorphic nucleotide corresponding thereto and AG for the polymorphic nucleotide at position 1316 of SEQ ID NO:17 or a polymorphic nucleotide corresponding thereto together indicates a favorable prognosis.

In some embodiments, the presence of at least one genotype selected from the group consisting of XRCC1 R194W CT and XRCC1 R399Q GG indicates an unfavorable prognosis.

In some embodiments, the presence of at least one genotype selected from the group consisting of CT for the polymorphic nucleotide at position 700 of SEQ ID NO:17 or a polymorphic nucleotide corresponding thereto and GG for the polymorphic nucleotide at position 1316 of SEQ ID NO:17 or a polymorphic nucleotide corresponding thereto indicates an unfavorable prognosis.

In some embodiments, the presence of XRCC1 R194W CT, and XRCC1 R399Q AG, or XRCC1 R399Q GG indicates an unfavorable prognosis.

In some embodiments, the presence of CT for the polymorphic nucleotide at position 700 of SEQ ID NO:17 or a polymorphic nucleotide corresponding thereto, and AG for the polymorphic nucleotide at position 1316 of SEQ ID NO:17 or a polymorphic nucleotide corresponding thereto, or GG for the polymorphic nucleotide at position 1316 of SEQ ID NO:17 or a polymorphic nucleotide corresponding thereto indicates an unfavorable prognosis

In some embodiments, the prognosis comprises overall survival of said subject.

In some embodiments, the favorable prognosis comprises an overall survival at least 1 year greater than the overall survival of an unfavorable prognosis. In some embodiments, the favorable prognosis comprises an overall survival at least 3 years greater than the overall survival of an unfavorable prognosis. In some embodiments, the favorable prognosis comprises an overall survival at least 6 years greater than the overall survival of an unfavorable prognosis.

In some embodiments, the treatment is selected from surgery, radiation therapy, proton therapy, chemotherapy, cryosurgery, and high intensity focused ultrasound. In some embodiments, the radiation therapy is selected from external beam radiotherapy and brachytherapy.

In some embodiments, the condition is castrate-resistant prostate cancer.

In some embodiments, the subject is human.

In some embodiments, the determining is performed in an automated device.

Some embodiments of the present invention include kits for evaluating a prognosis for radiation therapy in a subject with a prostate neoplastic condition comprising: at least one pair of oligonucleotides comprising sequences selected from the group consisting of SEQ ID NO:5 and SEQ ID NO:6, SEQ ID NO:7 and SEQ ID NO:8, SEQ ID NO:9 and SEQ ID NO:10, and SEQ ID NO:11 and SEQ ID NO:12. Some embodiments also include a tool for obtaining a sample from said subject. Some embodiments also include at least one reagent for isolating nucleic acids from an ex vivo sample taken from said subject. Some embodiments also include at least one reagent to perform a PCR. Some embodiments also include at least one reagent to perform nucleic acid sequencing.

Some embodiments of the present invention include kits for evaluating a response to radiation therapy in a subject with a prostate neoplastic condition comprising: a primer or probe which can be used to identify a genotype of the codon encoding amino acid 339 of the XRCC1 polypeptide; and a primer or probe which can be used to identify the genotype of the codon encoding amino acid 194 of the XRCC1 polypeptide. In some embodiments, the primer or probe which can be used to identify a genotype of the codon encoding amino acid 339 of the XRCC1 polypeptide can be used to identify a polymorphic nucleotide at position 1316 of SEQ ID NO:17 or a polymorphic nucleotide corresponding thereto, and said primer or probe which can be used to identify a genotype of the codon encoding amino acid 194 of the XRCC1 polypeptide can be used to identify a polymorphic nucleotide at position 700 of SEQ ID NO:17 or a polymorphic nucleotide corresponding thereto. Some embodiments also include a primer or probe which can be used to identify the genotype of the codon encoding amino acid 762 of the PARP1 polypeptide. In some embodiments, the primer or probe which can be used to identify a genotype of the codon encoding amino acid 762 of the PARP1 polypeptide can be used to identify a polymorphic nucleotide at position 2456 of SEQ ID NO:19 or a polymorphic nucleotide corresponding thereto.

Some embodiments of the present invention include methods for identifying one or more polymorphisms in the XRCC1 gene which is associated with a favorable or unfavorable response to radiation therapy in a subject having a prostate neoplastic condition comprising: determining the identity of one or more polymorphic nucleotides in the XRCC1 gene in a plurality of individuals having a prostate neoplastic condition who responded favorably to radiation therapy; determining the identity of one or more polymorphic nucleotides in the XRCC1 gene in a plurality of individuals having a prostate neoplastic condition who responded unfavorably to radiation therapy; and identifying one or more polymorphisms having a statistically significant correlation with a favorable response to radiation therapy. In some embodiments, the determining is performed in an automated device.

### BRIEF DESCRIPTION OF THE DRAWINGS

FIG. 1 shows a schematic diagram of the structure of the XRCC1 protein. The XRCC1 protein includes an N-terminus domain (NTD), a linker region, a nuclear localization signal domain (NLS), a first BRCA C-terminus domain (BRCT1), Ck2 phosphorylation sites (CK2), and a second BRCA C-terminus domain (BRCT2). The locations of the single nucleotide polymorphisms R194W and R399Q are indicated.

FIG.2 shows a graph of Kaplan-Meier curves for overall survival of all patients with castrate-resistant prostate cancer. Each curve represents patients with one of four haplotypes: XRCC1 R399Q AA/XRCC1 R194W CC; XRCC1 R399Q AG/XRCC1 R194W CC; XRCC1 R399Q AG/XRCC1 R194W CT; and XRCC1 R399Q GG/XRCC1 R194W CT.

FIG. 3 shows a graph of Kaplan-Meier curves for overall survival curve of patients with castrate-resistant prostate cancer who received radiotherapy. Each curve represents patients with one of four haplotypes: XRCC1 R399Q AA/XRCC1 R194W CC; XRCC1 R399Q AG/XRCC1 R194W CC; XRCC1 R399Q AG/XRCC1 R194W CT; and XRCC1 R399Q GG/XRCC1 R194W CT.

### DETAILED DESCRIPTION

Radiation therapy is a potentially curative, important treatment option in localized prostate cancer. However, at 8 years after radiation therapy, even in the best risk subset of patients, approximately 10% of patients will experience clinical disease recurrence. The identification of molecular markers of treatment success or failure may allow for the development of strategies to further improve treatment outcomes.

The present invention arises, in part, from the finding that particular genetic polymorphisms in the XRCC1 gene affected the outcome in patients who received radiotherapy for localized prostate cancer. Five molecular markers of DNA repair were analyzed in 513 patients with castrate-resistant prostate cancer, including 284 patients who received radiotherapy, 229 patients without radiotherapy, and 152 healthy individuals were genotyped for 5 polymorphisms in DNA excision repair genes: ERCC1 N118N (500C>T), XPD K751Q (2282A>C), XRCC1 R194W (685C>T), XRCC1 R399Q (1301G>A) and PARP1 V762A (2446T>C). The distribution of genetic polymorphisms in the patients with castrate-resistant prostate cancer and in healthy controls was compared, and the association between the polymorphisms and overall survival was investigated. In the radiation treated subgroup, the median survival time was associated with the XRCC1 haplotype. The median survival time was 11.75 years for patients with the XRCC1 R 399Q AA/R194W CC haplotype, 12.17 years for patients with the XRCC1 R399Q AG/R194W CC haplotype, 6.665 years for patients with the XRCC1 R399Q AG/R194W CT haplotype, and 6.21 years for patients with the XRCC1 R399Q GG/R194W CT haplotype (p=0.034). This association was not found when all patients were investigated. Accordingly, genetic polymorphisms in XRCC1 affect the outcome in patients who received radiotherapy for localized prostate cancer.

Some embodiments of the present invention include methods for evaluating a prognosis of a subject with a prostate neoplastic condition. Some such methods include obtaining a sample from the subject, and determining the presence of at least one marker in the sample, wherein at least one marker is selected from XRCC1 R399Q, XRCC1 R194W, and PARP1 V762A. Some such methods include determining the genotype of said subject at at least one codon selected from the group consisting of the codon encoding amino acid 399 of the XRCC1 polypeptide, the codon encoding amino acid 194 of the XRCC1 polypeptide, and the codon encoding amino acid 762 of the PARP1 polypeptide. In some such methods, the step of determining the genotype comprises determining the identity of a polymorphic nucleotide selected from the group consisting the nucleotide corresponding to nucleotide 1316 of SEQ ID NO:17, the nucleotide corresponding to nucleotide 700 of SEQ ID NO:17, and the nucleotide corresponding to nucleotide 2456 of SEQ ID NO:19.

Some embodiments of the present invention also include methods for evaluating a prognosis for radiation therapy in a subject with a prostate neoplastic condition. Some such methods include evaluating a prognosis for radiation therapy in the subject. In some embodiments of such methods, a sample is obtained from the subject. The presence of at least one marker in the sample is evaluated, wherein the at least one marker is selected from XRCC1 R399Q, XRCC1 R194W, and PARP1 V762A; and providing the result of the evaluating to a party in order for the party to select a treatment for the subject. Some embodiments of the present invention also include methods for selecting a treatment for a subject with a prostate neoplastic condition. In some embodiments of such methods, a sample is obtained from the subject. The presence of at least one marker in the sample is evaluated, wherein the at least one marker is selected from XRCC1 R399Q, XRCC1 R194W, and PARP1 V762A; and providing the result of the evaluating to a party in order for the party to select a treatment for the subject. Some such methods include evaluating a prognosis for radiation therapy in the subject and include obtaining a sample from the subject; and determining the presence of at least one marker in the sample, wherein at least one marker is selected from XRCC1 R399Q, XRCC1 R194W, and PARP1 V762A; and selecting a treatment for the subject. More embodiments of the present invention include kits for evaluating a prognosis for radiation therapy in a subject with a prostate neoplastic condition. Some such kits include at least one pair of oligonucleotides comprising sequences selected from SEQ ID NO:5 and SEQ ID NO:6, SEQ ID NO:7 and SEQ ID NO:8, SEQ ID NO:9 and SEQ ID NO:10, and SEQ ID NO:11 and SEQ ID NO:12.

Radiation therapy is an important treatment option for patients with localized, early stage prostate cancer. In patients with T1 to T3 lesions, without nodal or distant metastases, similar clinical results are obtained through surgery (radical prostatectomy) or radiation therapy. Radiation therapy can be delivered by any of several approaches: external beam, brachytherapy, and intensity modulated radiation therapy. However, with surgery or with radiation therapy, a percentage of patients with well-documented localized disease will experience the return of their malignancy.

In patients with low risk localized prostate cancer, treated with modern intensity modulated radiation therapy, actuarial prostate-specific antigen relapse-free survival is 85% to 89%. In unfavorable risk localized prostate cancer, the actuarial prostate-specific antigen relapse-free survival is 59% to 72% (DeVita Jr VT JL et al. Principles and Practice of Oncology. 8th ed: Lippincott Williams & Wilkins (LWW), 2008). Therefore, even in the group of patients with good clinical features and the favorable prognosis, 11% to 15% of these patients have intra-tumor characteristics that lead to relapse of disease. One question is whether there are intra-tumor considerations for DNA repair pathways that may make some prostate cancer cells more resistant to radiation therapy, and therefore make those tumors more likely to clinically recur. Though considerable inter-patient differences in response to radiotherapy occur, the mechanisms behind these different responses are not well understood.

A variety of factors contribute to the various outcomes of radiotherapy. Such factors include differences in patient, tumors, treatments, and molecular differences. The understanding of this mechanism may increase the predictability of outcome and selection of the optimal treatment. The work published by the Radiation Therapy Oncology Group investigated a total of 11 potential prognostic markers, and only p53 and DNA ploidy showed association with overall survival (Roach M, 3rd et al. A. Predictive models in external beam radiotherapy for clinically localized prostate cancer. Cancer. 2009 Jul. 1: 115:3112-20). Since ionizing radiation acts through creating various types of DNA damage, the inter-individual radiosensitivity may influence the patient's response to such therapy. The genetic polymorphisms in DNA repair genes may serve as the genetic basis for such inter-individual differences. Genetic polymorphisms in DNA repair genes are differently distributed in ethnic groups and might contribute to the ethnic disparity of sensitivity to DNA-damaging chemotherapy.

The types of DNA damage induced by radiation include DNA base damage and both single- and double-strand DNA breaks (Jorgensen T J. Enhancing radiosensitivity: targeting the DNA repair pathways. Cancer Biol Ther. 2009 April: 8:665-70). Such lesions, if inadequately repaired, can lead to cell death by lethal chromosomal aberrations or apoptosis, the desired outcome of radiation therapy. Multiple DNA repair pathways are involved to maintain the genomic integrity, and the homologous recombination and non-homologous end-joining, nucleotide excision repair (NER) and base excision repair (BER) pathways contribute heavily to remove the damage caused by ionizing radiation (Jorgensen T J. Enhancing radiosensitivity: targeting the DNA repair pathways. Cancer Biol Ther. 2009 April: 8:665-70; Hoeijmakers J H. Genome maintenance mechanisms for preventing cancer. Nature. 2001 May 17: 411:366-74).

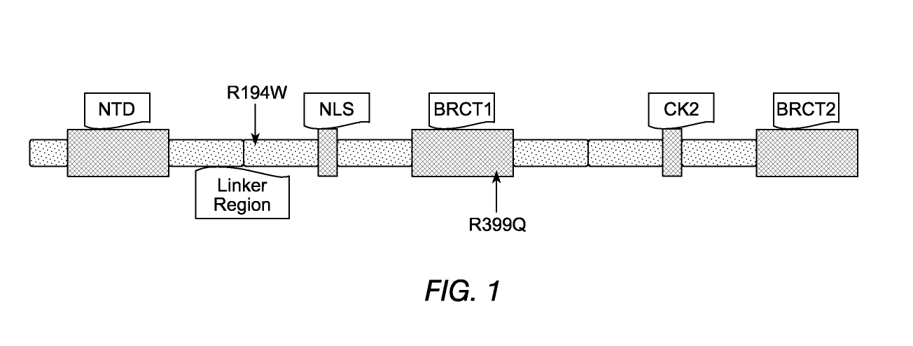

XRCC1 was the first human gene cloned in the BER pathway, and cells lacking this gene product are hypersensitive to ionizing radiation (Churchill M E et al. Repair of near-visible- and blue-light-induced DNA single-strand breaks by the CHO cell lines AA8 and EM9. Photochem Photobiol. 1991 October: 54:639-44). XRCC1 works as a stimulator and scaffold protein for other enzymes involved in this pathway. Polymorphisms have been identified in XRCC1 that correlate with phenotypic changes (Ladiges W C. Mouse models of XRCC1 DNA repair polymorphisms and cancer. Oncogene. 2006 Mar. 13: 25:1612-9). One important polymorphism in XRCC1 is R194W, located in the linker region separating the NH₂-terminal domain from the central BRCA1 C-terminus domain, as illustrated in FIG. 1. The linker region was also suggested to be a potential binding domain of several interactive proteins, and is rich in basic amino acids. The substitution of arginine to hydrophobic tryptophan may affect the protein binding efficiency. According to a review by Goode et al. (Goode E L et al. Polymorphisms in DNA repair genes and associations with cancer risk. Cancer Epidemiol Biomarkers Prey. 2002 December: 11:1513-30), the R194W polymorphism was related to reduced risk to cancer, and this was confirmed by two later association studies (Hu Z et al. XRCC1 polymorphisms and cancer risk: a meta-analysis of 38 case-control studies. Cancer Epidemiol Biomarkers Prey. 2005 July: 14:1810-8; Hung R J et al. Large-scale investigation of base excision repair genetic polymorphisms and lung cancer risk in a multicenter study. J Natl Cancer Inst. 2005 Apr. 20: 97:567-76). However, another study showed a highly significant association (p=0.0005) of R194W with the increased risk of head and neck cancer in a Korean population (Tae K et al. Association of DNA repair gene XRCC1 polymorphisms with head and neck cancer in Korean population. Int J Cancer. 2004 Sep. 20: 111:805-8). The second XRCC1 polymorphism, R399Q, is a well-studied single nucleotide polymorphism located in the BRCT1 domain, which is essential for PARP1 binding. Cells carrying this mutation have been shown to be defective in responding to both X-ray radiation and UV light (Au W W et al. Functional characterization of polymorphisms in DNA repair genes using cytogenetic challenge assays. Environ Health Perspect. 2003 November: 111:1843-50). Studies correlated the polymorphisms in XRCC1 with either adverse effects (Burri R J et al. Association of single nucleotide polymorphisms in SOD2, XRCC1 and XRCC3 with susceptibility for the development of adverse effects resulting from radiotherapy for prostate cancer. Radiat Res. 2008 July: 170:49-59) or protective effects resulting from radiotherapy (De Ruyck K et al. Radiation-induced damage to normal tissues after radiotherapy in patients treated for gynecologic tumors: association with single nucleotide polymorphisms in XRCC1, XRCC3, and OGG1 genes and in vitro chromosomal radiosensitivity in lymphocytes. Int J Radiat Oncol Biol Phys. 2005 Jul. 15: 62:1140-9; Chang-Claude J et al. Association between polymorphisms in the DNA repair genes, XRCC1, APE1, and XPD and acute side effects of radiotherapy in breast cancer patients. Clin Cancer Res. 2005 Jul. 1: 11:4802-9), or favorable response to therapeutic radiation in several cancers (Ho A Y et al. Genetic predictors of adverse radiotherapy effects: the Gene-PARE project. Int J Radiat Oncol Biol Phys. 2006 Jul. 1: 65:646-55).

PARP1, another important gene in DNA repair, assists by recruiting XRCC1 after sensing DNA damage. The variation, V762A in PARP1, causes the loss of two methyl groups that in turn increases the distance between 762 and its closest neighbor in the active site. This steric change loosens the binding of NAD⁺ and reduces the enzymatic activity nearly two fold (Wang X G et al. PARP1 Val762Ala polymorphism reduces enzymatic activity. Biochem Biophys Res Commun. 2007 Mar. 2: 354:122-6). As a consequence, the variant enzyme may be less able to sense the damage in DNA and reduces the recruitment of XRCC1 and other proteins involved in the repair process. Since PARP1 also plays an important role in repairing radiation inflicted lesions, several PARP1 inhibitors have been tested in clinical trials to try to increase the effectiveness of ionizing radiation in the treatment of cancer (Ben-Hur E. Involvement of poly (ADP-ribose) in the radiation response of mammalian cells. Int J Radiat Biol Relat Stud Phys Chem Med. 1984 December: 46:659-71; Arundel-Suto C M et al. Effect of PD 128763, a new potent inhibitor of poly(ADP-ribose) polymerase, on X-ray-induced cellular recovery processes in Chinese hamster V79 cells. Radiat Res. 1991 June: 126:367-71; Bowman K J et al. Potentiation of anti-cancer agent cytotoxicity by the potent poly(ADP-ribose) polymerase inhibitors NU1025 and NU1064. Br J Cancer. 1998 November: 78:1269-77.

In addition to BER, the NER pathway also plays a role in removing multiple types of DNA damage, including those caused by UV light and platinum-containing chemotherapy agents. Important genes in the NER, ERCC1, and XPF, are essential for the 5′ incision into the DNA strand that releases bulky DNA lesions (van Duin M et al. Molecular characterization of the human excision repair gene ERCC-1: cDNA cloning and amino acid homology with the yeast DNA repair gene RADIO. Cell. 1986 Mar. 28: 44:913-23; van Duin M et al. Genomic characterization of the human DNA excision repair gene ERCC-1. Nucleic Acids Res. 1987 Nov. 25: 15:9195-213). XPD is a 5′-3′ helicase that participates in DNA strand separation prior to the 5′ incision step performed by the ERCC1-XPF heterodimer (Sung P et al. Human xeroderma pigmentosum group D gene encodes a DNA helicase. Nature. 1993 Oct. 28: 365:852-5).

Laboratory studies indicated that the variant genotype of XRCC1 R399Q is more sensitive to X-ray and UV-light than the other two genotypes within this codon (Au W W et al. Functional characterization of polymorphisms in DNA repair genes using cytogenetic challenge assays. Environ Health Perspect. 2003 November: 111:1843-50). XRCC I R399Q is located in the BRCT1 domain (FIG. 1), a critical region that is required for PARP1 mediated recruitment of XRCC1 upon DNA damage. This site is involved in survival after methylation damage (Levy N et al. XRCC1 is phosphorylated by DNA-dependent protein kinase in response to DNA damage. Nucleic Acids Res. 2006: 34:32-41). Substitution of an arginine to glutamine could cause the loss of a secondary structure feature such as an alpha helix that is important for correct protein-protein interactions in the BRCT1 domain, and thus compromising the DNA repair capability (Monaco R et al. Conformational Effects of a Common Codon 399 Polymorphism on the BRCT1 Domain of the XRCC1 Protein. Protein J. 2007 Sep. 25). Patients possessing the variant genotype AA of the XRCC1 R399Q had a longer median survival (11.12 years comparing to 7.77 years and 8.17 years for the other two genotypes), although this was not statistically significant (p=0.5256). A study showed that the number of variant alleles in APE1 D148Q and XRCC1 R399Q genotypes was significantly correlated with prolonged cell-cycle delay following ionizing radiation, which resulted in ionizing radiation hypersensitivity in breast cancer cases (p=0.001) (Hu J J et al. Genetic regulation of ionizing radiation sensitivity and breast cancer risk. Environ Mol Mutagen. 2002: 39:208-15). Theoretically, the variant allele of the XRCC1 R399Q may impair the interaction between XRCC1 and other proteins, resulting in inefficient removal of radiation induced DNA damage and prolonged cell cycle arrest, which delivers favorable response to radiotherapy.

The polymorphism of R194W is located in a linker region (residues 158-310) between the NTD and the central BRCT domain of XRCC1 (FIG. 1), enriched in basic amino acids. The high pI and overall positive charge of this region was suggested to have an important role in proper secondary structure formation (Marintchev A et al. Domain specific interaction in the XRCC1-DNA polymerase beta complex. Nucleic Acids Res. 2000 May 15: 28:2049-59). This domain is also the potential protein-binding domain for several interactive protein partners (PCNA, APE1, etc.) of the XRCC1 protein. The transition from the positively charged arginine to a hydrophobic tryptophan could affect binding and DNA repair efficiency. An in silico study suggested that the presence of the variant allele of R194W might result in a damaging effect and an intolerant protein (Ladiges W C. Mouse models of XRCC1 DNA repair polymorphisms and cancer. Oncogene. 2006 Mar. 13: 25:1612-9). A low frequency of the variant genotype TT of this SNP was found in the study population described in EXAMPLE 1 (1% in the healthy volunteers and 2% in the patient group). In this patient group, the heterozygous genotype of the XRCC1 R194W was observed to tend to segregate from the variant homozygous genotype of R399Q, which may indicate that the wild type allele of R399Q has a protective effect that compensates the compromised protein function of XRCC1 caused by R194W allele. A previous study showed that the variant allele of R194W had higher frequency in radiation-sensitive breast cancer cases (OR 1.98, 95% CI 0.92-4.17) (Moullan N et al. Polymorphisms in the DNA repair gene XRCC1, breast cancer risk, and response to radiotherapy. Cancer Epidemiol Biomarkers Prey. 2003 November: 12:1168-74). The study described in EXAMPLE 1 also showed longer survival time in the patients with the variant genotype of R194W (9.22 years comparing to 8.06 years and 6.52 years) but not statistically significant (p=0.5493). However, in the haplotype analysis, as the result of it's tending to group with the wild type allele of XRCC1 R399Q, the variant allele of R194W showed a protective effect on radiotherapy. Though some epidemiological studies did suggest the variant allele of XRCC1 R194W confers reduced cancer risk (Goode EL et al. Polymorphisms in DNA repair genes and associations with cancer risk. Cancer Epidemiol Biomarkers Prey. 2002 December: 11:1513-30), others suggested vice versa (Tae K et al. Association of DNA repair gene XRCC1 polymorphisms with head and neck cancer in Korean population. Int J Cancer. 2004 Sep. 20: 111:805-8). The data presented in EXAMPLE 1 indicates that there may be a complicated intergenic interaction between the polymorphisms of XRCC1 R399Q and R194W. This intergenic interaction may be universal and extends to multiple DNA repair genes. Possessing more than 4 SNPs in DNA repair genes resulted in hypersensitivity to radiation in cells obtained from patients with cancer (p<0.001).

DNA repair pathways help to maintain genetic stability and prevent the development of cancer. However, they also represent a potential mechanism of resistance to DNA damaging chemotherapy and radiotherapy. The polymorphisms in DNA repair genes provide the genetic basis for various DNA repair capability. To identify radiosensitive cancer patients before treatment allows tailored radiotherapy and optimize the effectiveness and toxicity of ionizing radiation in clinical practice.

### Markers

Some embodiments of the present invention include methods and compositions to determine the presence of markers. Markers can include polymorphisms. As used herein, the term “polymorphism” refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A single nucleotide polymorphism is a single base pair change. Typically a single nucleotide polymorphism is the replacement of one nucleotide by another nucleotide at the polymorphic site. Deletion of a single nucleotide or insertion of a single nucleotide, also give rise to single nucleotide polymorphisms. In the context of the present invention “single nucleotide polymorphism” preferably refers to a single nucleotide substitution. Typically, between different genomes or between different individuals, the polymorphic site is occupied by two different nucleotides. In some embodiments, markers can include the genotype of a subject at a polymorphic site, for example, a marker can include the presence of a polymorphism in one, two, or more alleles at a polymorphic site in a subject's genome.

In some embodiments, markers include polymorphisms in a DNA repair gene. In some embodiments, markers include polymorphisms in a gene of the base excision repair pathway. In some embodiments, markers include polymorphisms in a gene of the nucleotide excision repair pathway. In some embodiments, markers include polymorphisms in the ERRC1, XPD, XRCC1, and PARP1 genes. Table 1 summarizes example markers. Each of the SNP identifiers set forth in Table 1 is incorporated herein by reference in its entirety and can be found in the NCBI database at http://www.ncbi.nlm.nih.gov/sites/snp. Each of the nucleic acid accession numbers set forth in Table 1 is incorporated by reference in its entirety. Each of the protein sequence accession numbers set forth in Table 1 is incorporated by reference in its entirety.

TABLE 1

| | | | | Example Nucleic | |
|---|---|---|---|---|---|
| | | Example | | Acid Accession | |
| | | Amino | Example | Number/ | |
| | | acid | Nucleotide | SNP Location in | Example Protein |
| Gene | SNP | change | changes | Nucleic Acid | Sequence |
| ERCC1 | rs11615 | N118N | A > T/C | NM_001166049.1 | NM_001166049.1 |
| | | | | (SEQ ID NO: 13)/500 | (SEQ ID NO: 14) |
| XPD/ | rs13181 | K751Q | A > C | NM_000400.3 (SEQ | NP_000391.1 (SEQ |
| ERCC2 | | | | ID NO: 15)/—; | ID NO: 16) |
| | | | | SEQ ID NO: 21/301 | |
| XRCC1 | rs25487 | R399Q | G > A | NM_006297.2 (SEQ | NP_006288.2 (SEQ |
| | | | | ID NO: 17)/1316 | ID NO: 18) |
| XRCC1 | rs1799782 | R194W | C > T | NM_006297.2(SEQ ID | NP_006288.2(SEQ |
| | | | | NO: 17)/700 | ID NO: 18) |
| PARP1 | rs1136410 | V762A | T > C | NM_001618.3(SEQ ID | NP_001609.2(SEQ |
| | | | | NO: 19)/2456 | ID NO: 20) |

For example, in some embodiments, a marker includes a SNP in ERCC1, such as rs11615; a SNP in the XPD/ERCC2 gene, such as rs13181; a SNP in the XRCC I gene, such as rs25487; a SNP in the XRCC1, such as rs1799782; a SNP in the PARP1 gene, such as rs1136410.

Some embodiments of the present invention involve determining the identity of a polymorphic nucleotide corresponding to the SNP locations in the nucleic acids listed in Table 1. For example, in some embodiments, the identity of the polymorphic marker corresponding to position 1316 in SEQ ID NO:17 is determined. In such as embodiment, the term “corresponding” relates to the fact that the location of the polymorphic nucleotide depends on the sequence of the nucleic acid utilized in the analysis, which can vary depending on the primers or techniques used to obtain the nucleic acid. For example, if a primer having a 5′ end which lies 20 nucleotides upstream of the 5′ end of SEQ ID NO:17 and a primer which is complementary to a sequence near the 3′ end of SEQ ID NO:17 and which hybridizes to SEQ ID NO:17 such that its 5′ terminal nucleotide is paired with the 3′ terminal nucleotide of 5′ are used in a PCR reaction, an application product having 20 additional nucleotides at its 5′ end relative to 5′ will be produced. In such an amplification product, the polymorphic nucleotide corresponding to nucleotide 1316 of SEQ ID NO:17 will be located at nucleotide number 1336. Thus, it will be appreciated that those skilled in the art can readily obtain nucleic acids in which the nucleotides corresponding to polymorphic nucleotides of the nucleic acids listed in Table 1 are located at various positions.

One skilled in the art can also use methods to align sequences are well known in the art and include, for example, algorithms and computer programs such as BLASTN, BLASTX, BLASTP, and the GCG Package of software (Wisconsin) to align nucleic acid s which completely or partially overlap with the nucleic acids or polypeptides listed in Table 1 and can identify the locations in the polymorphic nucleotides or amino acids within such overlapping sequences.

In some embodiments, a marker includes a polymorphic nucleotide in ERCC1, such as the nucleotide at 500 of SEQ ID NO:13, or a polymorphic nucleotide corresponding thereto; a polymorphic nucleotide in the XPD/ERCC2 gene, such as the nucleotide at 301 of SEQ ID NO:21, or a polymorphic nucleotide corresponding thereto; a polymorphic nucleotide in the XRCC1 gene, such as the nucleotide at 1316 of SEQ ID NO:17, or a polymorphic nucleotide corresponding thereto; a polymorphic nucleotide in the XRCC1, such as the nucleotide at 700 of SEQ ID NO:17, or a polymorphic nucleotide corresponding thereto; a polymorphic nucleotide in the PARPI gene, such as the nucleotide at 2456 of SEQ ID NO:19, or a polymorphic nucleotide corresponding thereto.

In some embodiments, a marker includes a codon encoding amino acid 399 of the XRCC1 polypeptide (e.g., the amino acid corresponding to position 399 of SEQ ID NO:18), the codon encoding amino acid 194 of the XRCC1 polypeptide (e.g., the amino acid corresponding to position 194 of SEQ ID NO:18), the codon encoding amino acid 762 of the PARP1 polypeptide (e.g., the amino acid corresponding to position 762 of SEQ ID NO:20), or the codon encoding amino acid 118 of the ERCC1 polypeptide (e.g., the amino acid corresponding to position 118 of SEQ ID NO:14).

In some embodiments, the markers include polymorphisms such as XRCC1 R399Q, XRCC1 R194W, and PARPI V762A. In some embodiments, markers can include the genotype of a subject at a polymorphic site, examples include, ERCC1 rs11615 CC; ERCC1 rs11615 CT; ERCC1 rs11615 TT; XPD rs13181 AA; XPD rs13181 AC; XPD rs13181 CC; XRCC1 rs1799782 CC; XRCC1 rs1799782 CT; XRCC1 rs1799782 TT; XRCC1 rs25487 GG; XRCC1 rs25487 GA; XRCC1 rs25487 AA; PARP1 rs1136410 TT; PARP1 rs1136410 TC; and PARP1 rs1136410 CC.

In some embodiments, the genotype of a subject at a polymorphic site can include, for example, the genotypes CC, CT, or TT at a nucleotide in ERCC1, such as the nucleotide at 500 of SEQ ID NO:13, or a polymorphic nucleotide corresponding thereto; the genotypes AA, AC, or CC at a nucleotide in the XPD/ERCC2 gene, such as the nucleotide at 301 of SEQ ID NO:21, or a polymorphic nucleotide corresponding thereto; the genotypes AA, GG, or GA, at a nucleotide in the XRCC1 gene, such as the nucleotide at 1316 of SEQ ID NO:17, or a polymorphic nucleotide corresponding thereto; the genotypes CC, TT, or CT at a nucleotide in the XRCC1, such as the nucleotide at 700 of SEQ ID NO:17, or a polymorphic nucleotide corresponding thereto; and the genotype TT, CC, or CT at a nucleotide in the PARP1 gene, such as the nucleotide at 2456 of SEQ ID NO:19, or a polymorphic nucleotide corresponding thereto.

### Marker Detection

In some embodiments, the presence of polymorphisms in a sample may be determined by sequencing nucleic acid, e.g., DNA, RNA, and cDNA, or an amplified region thereof, obtained from a subject. As used herein, the term “subject” includes any animal, including a mammal such as a human, dog, cat, mouse, horse, or primate.

In some embodiments, nucleic acid may be extracted from a subject's biological sample using any appropriate method. As used herein, the term “sample” refers to any biological fluid, cell, tissue, organ or portion thereof, e.g., blood, a biopsy of a tumor. The sample can comprise an in vivo sample or an ex vivo sample.

In some embodiments, a nucleic acid may be amplified and the product may then be purified, for example by gel purification, and the resulting purified product may be sequenced. Examples of methods for determining sequence information of nucleic acids include the dideoxy termination method of Sanger (Sanger et al., Proc. Natl. Acad. Sci. U.S.A. 74: 563-5467 (1977)); the Maxam-Gilbert chemical degradation method (Maxam and Gilbert, Proc. Natl. Acad. Sci. U.S.A. 74: 560-564 (1977)); Sanger-extension method using dyes associated with terminal nucleotides, gel electrophoresis and automated fluorescent detection; techniques using mass spectroscopy instead of electrophoresis; pyrophosphate release techniques (Ronaghi et al., Science 281: 363-365 (1998) and Hyman, Anal. Biochem. 174: 423-436 (1988)); single molecule sequencing techniques utilizing exonucleases to sequentially release individual fluorescently labeled bases (Goodwin et al., Nucleos. Nucleot. 16: 543-550 (1997)); techniques pulling DNA through a thin liquid film as it is digested in order to spatially separate the cleaved nucleotides (Dapprich et al., Bioimaging 6: 25-32 (1998)); techniques determining the spatial sequence of fixed and stretched DNA molecules by scanned atomic probe microscopy (Hansma et al., Science 256: 1180-1184 (1992)); techniques described in U.S. Pat. No. 5,302,509 to Cheeseman and in U.S. 2003/0044781 (Korlach); and techniques using hybridization of (substantially) complementary probes as described, e.g., in U.S. Pat. Publication Nos. 2005/0142577 and 2005/0042654.

Suitable amplification reactions include the polymerase chain reaction (PCR) (reviewed for instance in “PCR protocols; A Guide to Methods and Applications”, Eds. Innis et al, 1990, Academic Press, New York, Mullis et al, Cold Spring Harbor Symp. Quant. Biol., 51:263, (1987), Ehrlich (ed), PCR technology, Stockton Press, NY, 1989, and Ehrlich et al, Science, 252:1643-1650, (1991)).

In some embodiments, a marker can be detected utilizing allele-specific amplification methods which can discriminate between two alleles of a polymorphic nucleotide. In some such methods, one of the alleles is amplified without amplification of the other allele. This is accomplished by placing the polymorphic base at the 3′ end of one of the amplification primers. Because the extension forms from the 3′ end of the primer, a mismatch at or near this position has an inhibitory effect on amplification. Therefore, under appropriate amplification conditions, these primers only direct amplification on their complementary allele. Designing the appropriate allele-specific primer and the corresponding assay conditions are well with the ordinary skill in the art.

Other methods which are particularly suited for the detection of markers such as single nucleotide polymorphisms include LCR (ligase chain reaction). LCR uses two pairs of probes to exponentially amplify a specific target. The sequences of each pair of oligonucleotides, is selected to permit the pair to hybridize to abutting sequences of the same strand of the target. Such hybridization forms a substrate for a template-dependant ligase. LCR can be performed with oligonucleotides having the proximal and distal sequences of the same strand of a polymorphic nucleotide. In one embodiment, either oligonucleotide will be designed to include the polymorphic nucleotide. In such an embodiment, the reaction conditions are selected such that the oligonucleotides can be ligated together only if the target molecule either contains or lacks the specific nucleotide that is complementary to the polymorphic nucleotide on the oligonucleotide. In an alternative embodiment, the oligonucleotides will not include the polymorphic nucleotide, such that when they hybridize to the target molecule, a “gap” is created as described in WO 90/01069. This gap is then “filled” with complementary dNTPs (as mediated by DNA polymerase), or by an additional pair of oligonucleotides. Thus at the end of each cycle, each single strand has a complement capable of serving as a target during the next cycle and exponential allele-specific amplification of the desired sequence is obtained.

In some embodiments, a marker can be detected utilizing hybridization assay methods. A preferred method of determining the identity of the nucleotide present at a polymorphic nucleotide involves nucleic acid hybridization. The hybridization probes, which can be conveniently used in such reactions, preferably include the probes to sequences that include markers described herein. Any hybridization assay is used including Southern hybridization, Northern hybridization, dot blot hybridization and solid-phase hybridization (see Sambrook et al., Molecular Cloning—A Laboratory Manual, Second Edition, Cold Spring Harbor Press, N.Y., 1989).

Hybridization refers to the formation of a duplex structure by two single stranded nucleic acids due to complementary base pairing. Hybridization can occur between exactly complementary nucleic acid strands or between nucleic acid strands that contain minor regions of mismatch. Specific probes can be designed that hybridize to one form of a polymorphic nucleotide and not to the other and therefore are able to discriminate between different allelic forms.

Allele-specific probes are often used in pairs, one member of a pair showing perfect match to a target sequence containing the original allele and the other showing a perfect match to the target sequence containing the alternative allele. Hybridization conditions should be sufficiently stringent that there is a significant difference in hybridization intensity between alleles, and preferably an essentially binary response, whereby a probe hybridizes to only one of the alleles. Stringent, sequence specific hybridization conditions, under which a probe will hybridize only to the exactly complementary target sequence are well known in the art (Sambrook et al., Molecular Cloning—A Laboratory Manual, Second Edition, Cold Spring Harbor Press, N.Y., 1989). Stringent conditions are sequence dependent and will be different in different circumstances. Generally, stringent conditions are selected to be about 5° C. lower than the thermal melting point T_{M} for the specific sequence at a defined ionic strength and pH. By way of example and not limitation, procedures using conditions of high stringency are as follows: Prehybridization of filters containing DNA is carried out for 8 h to overnight at 65° C. in buffer composed of 6×SSC, 50 mM Tris-HCl (pH 7.5),1 mM EDTA, 0.02% PVP, 0.02% Ficoll, 0.02% BSA, and 500 μg/ml denatured salmon sperm DNA. Filters are hybridized for 48 h at 65° C., the preferred hybridization temperature, in prehybridization mixture containing 100 μg/ml denatured salmon sperm DNA and 5-20 .times.10⁶ cpm of ³²P-labeled probe. Alternatively, the hybridization step can be performed at 65° C. in the presence of SSC buffer, 1×SSC corresponding to 0.15 M NaCl and 0.05 M Na citrate. Subsequently, filter washes can be done at 37° C. for 1 h in a solution containing 2×SSC, 0.01% PVP, 0.01% Ficoll, and 0.01% BSA, followed by a wash in 0.1×SSC at 50° C. for 45 min. Alternatively, filter washes can be performed in a solution containing 2×SSC and 0.1% SDS, or 0.5×SSC and 0.1% SDS, or 0.1×SSC and 0.1% SDS at 68° C. for 15 minute intervals. Following the wash steps, the hybridized probes are detectable by autoradiography. By way of example and not limitation, procedures using conditions of intermediate stringency are as follows: Filters containing DNA are prehybridized, and then hybridized at a temperature of 60° C. in the presence of a 5×SSC buffer and labeled probe. Subsequently, filters washes are performed in a solution containing 2×SSC at 50° C. and the hybridized probes are detectable by autoradiography. Other conditions of high and intermediate stringency which is used are well known in the art and as cited in Sambrook et al. (Molecular Cloning—A Laboratory Manual, Second Edition, Cold Spring Harbor Press, N.Y., 1989) and Ausubel et al. (Current Protocols in Molecular Biology, Green Publishing Associates and Wiley Interscience, N.Y., 1989).

Although such hybridizations can be performed in solution, it is preferred to employ a solid-phase hybridization assay. The target DNA comprising a polymorphic nucleotide is amplified prior to the hybridization reaction. The presence of a specific allele in the sample is determined by detecting the presence or the absence of stable hybrid duplexes formed between the probe and the target DNA. The detection of hybrid duplexes can be carried out by a number of methods. Various detection assay formats are well known which utilize detectable labels bound to either the target or the probe to enable detection of the hybrid duplexes. Typically, hybridization duplexes are separated from unhybridized nucleic acids and the labels bound to the duplexes are then detected. Those skilled in the art will recognize that wash steps is employed to wash away excess target DNA or probe. Standard heterogeneous assay formats are suitable for detecting the hybrids using the labels present on the primers and probes.

Two recently developed assays allow hybridization-based allele discrimination with no need for separations or washes (see Landegren U. et al., Genome Research, 8:769-776,1998). The TaqMan assay takes advantage of the 5′ nuclease activity of Taq DNA polymerase to digest a DNA probe annealed specifically to the accumulating amplification product. TaqMan probes are labeled with a donor-acceptor dye pair that interacts via fluorescence energy transfer. Cleavage of the TaqMan probe by the advancing polymerase during amplification dissociates the donor dye from the quenching acceptor dye, greatly increasing the donor fluorescence. All reagents necessary to detect two allelic variants can be assembled at the beginning of the reaction and the results are monitored in real time (see Livak et al., Nature Genetics, 9:341-342, 1995). In an alternative homogeneous hybridization based procedure, molecular beacons are used for allele discriminations. Molecular beacons are hairpin-shaped oligonucleotide probes that report the presence of specific nucleic acids in homogeneous solutions. When they bind to their targets they undergo a conformational reorganization that restores the fluorescence of an internally quenched fluorophore (Tyagi et al., Nature Biotechnology, 16:49-53, 1998).

The polynucleotides provided herein or portions thereof can be used as probes in hybridization assays for the detection of polymorphic nucleotides in biological samples. These probes are characterized in that they preferably comprise between 8 and 50 nucleotides, and in that they are sufficiently complementary to a sequence comprising a polymorphic nucleotide described herein to hybridize thereto and preferably sufficiently specific to be able to discriminate the targeted sequence for only one nucleotide variation. The GC content in the probes usually ranges between 10 and 75%, preferably between 35 and 60%, and more preferably between 40 and 55%. The length of these probes can range from 10, 15, 20, or 30 to at least 100 nucleotides, preferably from 10 to 50, more preferably from 18 to 35 nucleotides. A particularly preferred probe is 25 nucleotides in length. Preferably the polymorphic nucleotide is within 4 nucleotides of the center of the polynucleotide probe. In particularly preferred probes the polymorphic nucleotide is at the center of said polynucleotide. Shorter probes may lack specificity for a target nucleic acid sequence and generally require cooler temperatures to form sufficiently stable hybrid complexes with the template. Longer probes are expensive to produce and can sometimes self-hybridize to form hairpin structures. Methods for the synthesis of oligonucleotide probes are well known in the art and can be applied to the probes of the present invention.

Preferably the probes described herein are labeled or immobilized on a solid support. Detection probes are generally nucleic acid sequences or uncharged nucleic acid analogs such as, for example peptide nucleic acids which are disclosed in International Patent Application WO 92/20702, morpholino analogs which are described in U.S. Pat. Nos. 5,185,444; 5,034,506 and 5,142,047. The probe may have to be rendered “non-extendible” in that additional dNTPs cannot be added to the probe. In and of themselves analogs usually are non-extendible and nucleic acid probes can be rendered non-extendible by modifying the 3′ end of the probe such that the hydroxyl group is no longer capable of participating in elongation. For example, the 3′ end of the probe can be functionalized with the capture or detection label to thereby consume or otherwise block the hydroxyl group. Alternatively, the 3′ hydroxyl group simply can be cleaved, replaced or modified, U.S. patent application Ser. No. 07/049,061 filed Apr. 19, 1993 describes modifications, which can be used to render a probe non-extendible.

The probes described herein are useful for a number of purposes. They can be used in Southern hybridization to genomic DNA or Northern hybridization to mRNA. The probes can also be used to detect PCR amplification products. By assaying the hybridization to an allele specific probe, one can detect the presence or absence of a polymorphic allele in a given sample.

High-Throughput parallel hybridizations in array format are specifically encompassed within “hybridization assays” and are described herein, for example, hybridization to addressable arrays of oligonucleotides. Hybridization assays based on oligonucleotide arrays rely on the differences in hybridization stability of short oligonucleotides to perfectly matched and mismatched target sequence variants. Efficient access to polymorphism information is obtained through a basic structure comprising high-density arrays of oligonucleotide probes attached to a solid support (the chip) at selected positions. Each DNA chip can contain thousands to millions of individual synthetic DNA probes arranged in a grid-like pattern and miniaturized to the size of a dime.

The chip technology has already been applied with success in numerous cases. For example, the screening of mutations has been undertaken in the BRCA1 gene, in S. cerevisiae mutant strains, and in the protease gene of HIV-1 virus (Hacia et al., Nature Genetics, 14(4):441-447, 1996; Shoemaker et al., Nature Genetics, 14(4):450-456, 1996; Kozal et al., Nature Medicine, 2:753-759, 1996). Chips of various formats for use in detecting polymorphisms can be produced on a customized basis by Affymetrix (GeneChip™), Hyseq (HyChip and HyGnostics), and Protogene Laboratories.

In general, these methods employ arrays of oligonucleotide probes that are complementary to target nucleic acid sequence segments from an individual which, target sequences include a polymorphic marker. European Patent No. 785280 describes a tiling strategy for the detection of single nucleotide polymorphisms. Briefly, arrays may generally be “tiled” for a large number of specific polymorphisms. By “tiling” is generally meant the synthesis of a defined set of oligonucleotide probes which is made up of a sequence complementary to the target sequence of interest, as well as preselected variations of that sequence, e.g., substitution of one or more given positions with one or more members of the basis set of monomers, i.e. nucleotides. Tiling strategies are further described in PCT application No. WO 95/11995. In a particular aspect, arrays are tiled for a number of specific, identified polymorphic nucleotide sequences. In particular the array is tiled to include a number of detection blocks, each detection block being specific for a specific polymorphic nucleotide or a set of polymorphic nucleotides. For example, a detection block is tiled to include a number of probes, which span the sequence segment that includes a specific polymorphism. To ensure probes that are complementary to each allele, the probes are synthesized in pairs differing at the polymorphic nucleotide. In addition to the probes differing at the polymorphic base, monosubstituted probes are also generally tiled within the detection block. These monosubstituted probes have bases at and up to a certain number of bases in either direction from the polymorphism, substituted with the remaining nucleotides (selected from A, T, G, C and U). Typically the probes in a tiled detection block will include substitutions of the sequence positions up to and including those that are 5 bases away from the polymorphic nucleotide. The monosubstituted probes provide internal controls for the tiled array, to distinguish actual hybridization from artifactual cross-hybridization. Upon completion of hybridization with the target sequence and washing of the array, the array is scanned to determine the position on the array to which the target sequence hybridizes. The hybridization data from the scanned array is then analyzed to identify which allele or alleles of the polymorphic nucleotide are present in the sample. Hybridization and scanning is carried out as described in PCT application No. WO 92/10092 and WO 95/11995 and U.S. Pat. No. No. 5,424,186.

Another technique, which is used to analyze polymorphisms, includes multicomponent integrated systems, which miniaturize and compartmentalize processes such as PCR and capillary electrophoresis reactions in a single functional device. An example of such technique is disclosed in U.S. Pat. No. 5,589,136, which describes the integration of PCR amplification and capillary electrophoresis in chips. Integrated systems can be envisaged mainly when microfluidic systems are used. These systems comprise a pattern of microchannels designed onto a glass, silicon, quartz, or plastic wafer included on a microchip. The movements of the samples are controlled by electric, electroosmotic or hydrostatic forces applied across different areas of the microchip to create functional microscopic valves and pumps with no moving parts. Varying the voltage controls the liquid flow at intersections between the micro-machined channels and changes the liquid flow rate for pumping across different sections of the microchip. For genotyping polymorphic nucleotides, the microfluidic system may integrate nucleic acid amplification, microsequencing, capillary electrophoresis and a detection method such as laser-induced fluorescence detection.

In some embodiments, the presence of a marker may be determined at the protein level by detecting the presence of a variant (i.e. a mutant or allelic variant) polypeptide. For example, antibodies that recognize a specific allele can be used to determine the presence of a particular marker.

Methods for Prognosis

Some embodiments of the present invention include methods for evaluating a prognosis of a subject with a prostate neoplastic condition. As used herein, “prognosis” can refer to a predicted outcome of a condition. In some embodiments, the predicted outcome can include a determination in view of a particular treatment that a subject may receive, a particular treatment that a subject may continue to receive, or lack of a particular treatment. In some embodiments, the predicted outcome can include, for example, the survival of a subject. The survival of a subject can include, for example, the overall survival of a subject, and/or the survival of a subject free of a condition.

As used herein, the term “prostate neoplastic condition” refers to any condition that contains neoplastic prostate cells. Prostate neoplastic conditions include, for example, prostate interepithelial neoplasia and prostate cancer. Prostate cancer is an uncontrolled proliferation of prostate cells which can invade and destroy adjacent tissues as well as metastasize. Primary prostate tumors can be sorted into stages using classification systems such as the Gleason score. The Gleason score evaluates the degree of differentiation of the cells in a sample. A lower score (such as 1, 2, 3 or 4) indicates that the cells in the sample are differentiated and fairly normal looking, moderate scores such as 5, 6, or 7 indicate that the cells are moderately differentiated, and higher scores such as 8, 9, or 10 indicate poorly differentiated cells. The stage of overall disease, for example, for prostate cancer can be accessed using staging systems such as the Jewett-Whitmore system or the tumor, node, metastases system. The Jewett system classifies prostate cancer into one of four stages distinguished by the letters A, B, C, and D. Subdivisions that reflect specific conditions within each category can also be added to the Jewett system and this expanded alphanumeric system is called the Jewett-Whitmore system. The tumor, node, metastases system uses stages generally similar to those of the Jewett-Whitmore system but with expanded alphanumeric subcategories to describe primary tumors, regional lymph node involvement or distant metastasis. Similarly, there are classifications known by those skilled in the art for the progressive stages of precancerous lesions or prostate interepithelial neoplasia. The methods and compositions described herein are applicable for the diagnosis or prognosis of any or all stages of prostate neoplastic conditions. In particular embodiments, the prostate neoplastic condition includes castrate-resistant prostate cancer.

In some methods of prognosis, the presence or absence of a particular marker or combination of markers can be used to evaluate a favorable or unfavorable prognosis. In some embodiments, a favorable prognosis can include the increased survival of a subject receiving a particular treatment or a subject that would receive a particular treatment. In some embodiments, the treatment may be radiation therapy. In some embodiments, the increased survival of a subject can be relative to a subject that would receive no treatment or an alternative treatment. In some embodiments, an increased survival can include at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, and at least about 24 months. In some embodiments, an increased survival can include at least about 1 year, at least about 2 years, at least about 3 years, at least about 4 years, at least about 5 years, at least about 6 years, at least about 7 years, at least about 8 years, at least about 9 years, and at least about 10 years.

In some embodiments, an unfavorable prognosis can include the decreased survival of a subject receiving no treatment or a subject that would receive a particular treatment. In some embodiments, the decreased survival of a subject can be relative to a subject that would receive an alternative treatment. In some embodiments, a decreased survival can include at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, and at least about 24 months. In some embodiments, a decreased survival can include at least about 1 year, at least about 2 years, at least about 3 years, at least about 4 years, at least about 5 years, at least about 6 years, at least about 7 years, at least about 8 years, at least about 9 years, and at least about 10 years.

In some embodiments, a prognosis can be evaluated steps comprising determining the presence or absence of at least one marker described herein, or a combination of markers described herein. In some embodiments, at least one marker to evaluate a prognosis can include one or more of the markers described herein. In some embodiments, the genotype of a subject can be used to evaluate a prognosis.

In some embodiments, a favorable prognosis can be indicated by the presence of at least one marker and corresponding genotype including XRCC1 R399Q AA, PARPI V762A CC, and XRCC1 R194W CC. In some embodiments, a favorable prognosis can be indicated by the presence of a combination of markers and corresponding genotype such as XRCC1 R194W CC and XRCC1 R399Q AA, and XRCC1 R194W CC and XRCC I R399Q AG.

In some embodiments, a favorable prognosis can be indicated by the presence of at least one marker and corresponding genotype including the genotype AA, at a nucleotide in the XRCC1 gene, such as the nucleotide at 1316 of SEQ ID NO:17, and nucleotide corresponding thereto; the genotype CC at a nucleotide in the XRCC1, such as the nucleotide at 700 of SEQ ID NO:17, and nucleotide corresponding thereto; and the genotype CC at a nucleotide in the PARP1 gene, such as the nucleotide at 2456 of SEQ ID NO:19, and nucleotide corresponding thereto.

In some embodiments, a favorable prognosis can be indicated by the combination of the genotype AA at a nucleotide in the XRCC1 gene, such as the nucleotide at 1316 of SEQ ID NO:17, and nucleotide corresponding thereto, and the genotype CC at a nucleotide in the XRCC1 gene, such as the nucleotide at 700 of SEQ ID NO:17, and nucleotide corresponding thereto. In some embodiments, a favorable prognosis can be indicated by the combination of the genotype AG at a nucleotide in the XRCC1 gene, such as the nucleotide at 1316 of SEQ ID NO:17, and nucleotide corresponding thereto, and the genotype CC at a nucleotide in the XRCC1, such as the nucleotide at 700 of SEQ ID NO:17, and nucleotide corresponding thereto.

In some embodiments, an unfavorable prognosis can be indicated by the presence of at least one marker including XRCC1 R194W CT and XRCC1 R399Q GG. In some embodiments, an unfavorable prognosis can be indicated by the presence of a combination of markers such as XRCC1 R194W CT and XRCC1 R399Q GG, and XRCC1 R194W CT and XRCC1 R399Q AG.

In some embodiments, an unfavorable prognosis can be indicated by the presence of at least one marker and corresponding genotype including the genotype GG, at a nucleotide in the XRCC1 gene, such as the nucleotide at 1316 of SEQ ID NO:17, and nucleotide corresponding thereto; and the genotype CT at a nucleotide in the XRCC1, such as the nucleotide at 700 of SEQ ID NO:17, and nucleotide corresponding thereto.

In some embodiments, an unfavorable prognosis can be indicated by the combination of genotype GG at a nucleotide in the XRCC1 gene, such as a nucleotide corresponding to the nucleotide at 1316 of SEQ ID NO:17, and nucleotide corresponding thereto, and the genotype CT at a nucleotide in the XRCC1, such as the nucleotide at 700 of SEQ ID NO:17, and nucleotide corresponding thereto. In some embodiments, a favorable prognosis can be indicated by the combination of the genotype AG at a nucleotide in the XRCC1 gene, such as the nucleotide at 1316 of SEQ ID NO:17, and nucleotide corresponding thereto, and the genotype CT at a nucleotide in the XRCC1, such as the nucleotide at 700 of SEQ ID NO:17, and nucleotide corresponding thereto.

Determining Methods of Treatment

Some embodiments of the present invention include methods of treating a subject with a neoplastic prostate condition. Some embodiments include selecting a particular treatment for a subject. The selection of a particular treatment can be determined in view of a subject's favorable or unfavorable prognosis for the particular treatment. For example, a favorable prognosis for a subject that would be treated with radiation therapy can be used to determine that the treatment for the subject should include radiation therapy. Conversely, an unfavorable prognosis for a subject that would be treated with radiation therapy or can be used to determine that the treatment for the subject should not be radiation therapy and an alternative treatment should be administered to the subject. The selection of a particular treatment or combination of treatments may be evaluated by the methods provided herein and can include an evaluation of factors such as the stage of neoplastic prostate condition, the Gleason score, the subject's age, the subject's general health.

Some methods for determining a method of treating a subject include providing information to a party in order for the party to select a particular treatment for a subject. As used herein, “party” can refer to an entity receiving information from another entity. An example of a party can include a care-giver, care-provider, and physician. In some embodiments, the information can include a determination of the presence or absence of markers described herein. In some embodiments, the information can include an evaluation of a prognosis for a subject. In some embodiments, a party receiving information can evaluate a prognosis of a subject in view of the information received by the party. In some embodiments, a party receiving information can select a treatment for a subject in view of the information received by the party.

Several treatment options are available for subjects with prostate neoplastic conditions. Examples of treatments include radiation therapy, active surveillance, surgery, and hormone therapy. The methods and compositions described herein can be used to determine an appropriate treatment for a particular subject to increase the survival of the subject.

Radiation therapy uses high energy rays to kill cancer cells and shrink tumors. It is often used when cancer cells are found in more than one area. Impotence can occur in subjects treated with radiation therapy. Two types of radiation therapy are used to treat prostate cancer: brachytherapy and external beam radiation therapy. Brachytherapy is the implantation of tiny, radioactive implants into a cancerous prostate gland. Radiation emitted by the implants kills the malignant tumor. External beam radiation therapy delivers a higher and more focused dose of radiation with fewer side effects and at lower cost than external beam therapy. Surgery usually removes the entire prostate and surrounding tissues (called a radical prostatectomy). Impotence and incontinence are possible side effects of surgery. Another kind of surgery is a transurethral resection, which cuts cancer from the prostate but does not take out the entire prostate. This operation is sometimes done to relieve symptoms caused by the tumor before other treatment or in subjects who cannot have a radical prostatectomy. Active surveillance is one of the most conservative treatment options. Subjects may have regular checkups so they can be closely monitored by a care-provider. A risk associated with active surveillance is that a subject can have a prostate neoplastic disease that grows rapidly or suddenly between checkups.

More examples of methods of treatment for a subject with a neoplastic prostate condition include high-intensity focused ultrasound, proton therapy, cryosurgery, chemotherapy, or some combination of the treatments described herein and/or those known in the art. High-intensity focused ultrasound is a precise medical procedure using a high-intensity focused ultrasound medical device to heat and destroy pathogenic tissue rapidly. This treatment is administered through a trans-rectal probe and relies on heat developed by focusing ultrasound waves into the prostate to kill the tumor. Proton therapy is a type of particle therapy which uses a beam of protons to irradiate diseased tissue. During treatment, a particle accelerator is used to target the tumor with a beam of protons. These charged particles damage the DNA of cells, ultimately causing their death or interfering with their ability to reproduce. Cancerous cells, because of their high rate of division and their reduced ability to repair damaged DNA, are particularly vulnerable to attack on their DNA Chemotherapy can include treatment with compounds such as bevacizumab, taxotere, thalidomide and prednisone, provenge, and cabazitaxel.

### Kits

Some embodiments of the present invention include kits. Some kits can be used to evaluate a prognosis in a subject with a prostate neoplastic condition. In some embodiments, kits can be used to determine the presence of particular markers in a sample obtained from a subject. Markers include at least any marker described herein.

Some kits can include oligonucleotides to determine the presence of particular markers described herein. Such oligonucleotides can be used to amplify nucleic acids of a sample obtained from a subject. For example, some kits can include at least one pair of oligonucleotides comprising sequences including SEQ ID NO:5 and SEQ ID NO:6, SEQ ID NO:7 and SEQ ID NO:8, SEQ ID NO:9 and SEQ ID NO:10, and SEQ ID NO:11 and SEQ ID NO:12. Some kits can include at least one oligonucleotide that include sequences including SEQ ID NO:1-12. Some kits can include oligonucleotides to sequence a nucleic acid of a sample obtained from a sample in order to determine the presence or absence of a particular marker described herein.

Some kits can include a tool for obtaining a sample from a subject. For example, a swab to obtain a cheek cell sample, a mouthwash to obtain a cheek cell sample, a needle and syringe to obtain fluid samples such as blood, or a punch tool to obtain a punch-biopsy. Some kits can include at least one reagent for isolating nucleic acids from a sample taken from the subject. Some kits can include at least one reagent to perform a PCR, for example a polymerase, such as a thermostable polymerase, and nucleotides. Some kits can include at least one reagent to perform nucleic acid sequencing, for example, a polymerase and nucleotides. Some kits can include instructions for use of such kits. Instructions can include evaluating the results of determining the presence of particular markers in a sample

### EXAMPLES

### Example 1

### Association Between Polymorphisms in NER and BER DNA Repair Genes and Clinical Outcome of Radiotherapy in Patients with Prostate Cancer

This study investigated the association between polymorphisms in NER and BER DNA repair genes and clinical outcome of radiotherapy in patients with prostate cancer. Five hundred and thirteen patients with castrate-resistant prostate cancer were analyzed, including 284 patients who received external beam radiotherapy (XRT) and/or brachytherapy, and 229 patients who did not receive radiotherapy. All patients were Caucasians. A control group included 152 male Caucasian subjects with no diagnosis of cancer.

Genomic DNA was extracted from serum or white blood cell buffy coat layers of whole blood of patients, or NCI-60 cell pellets (Hamada A et al. Urology. 2007 August: 70:217-20). Polymerase chain reaction (PCR) and direct nucleotide sequencing were performed (Gao R et al. Ethnic disparities in Americans of European descent versus Americans of African descent related to polymorphic ERCC1, ERCC2, XRCC1, and PARP1. Mol Cancer Ther. 2008 May: 7:1246-50). Table 2 shows oligonucleotide primers used in the analysis.

TABLE 2

| | | | Oligo | | |
|---|---|---|---|---|---|
| | | Sequence | Start | | |
| | | including | position | | |
| | | amplified | in | | Product |
| SNP | Oligo | Sequence | Sequence | Oligo Sequence | length |
| ERCC1 | F1 | | | (SEQ ID NO: 01) | 542 |
| N118N | | | | TGGATCAGAGGATCAGGGAC | |
| (rs11615) | R1 | | | (SEQ ID NO: 02) | |
| | | | | TTCCTGAGACCCAGGAGTTC | |
| XPD | F1 | SEQ ID | 122 | (SEQ ID NO: 03) | 415 |
| K751Q | | NO: 21 | | CCTTCTCCTGCGATTAAAGGCTGT | |
| (rs13181) | R1 | SEQ ID | 536 | (SEQ ID NO: 04) | |
| | | NO: 21 | | TCAGCCCCATCTTATGTTGACAGG | |
| XRCC1 | F1 | | | (SEQ ID NO: 05) | |
| R399Q | | | | AGACAAAGATGAGGCAGAGG | |
| (rs25487) | R1 | | | (SEQ ID NO: 06) | |
| | | | | TCAACCCTCAGGACACAAGAG | |
| XRCC1 | F1 | | | (SEQ ID NO: 07) | |
| R194W | | | | TGCATCTCTCCCTTGGTCTCC | |
| (rs1799782) | R1 | | | (SEQ ID NO: 08) | |
| | | | | TGCACAAACTGCTCCTCCAGC | |
| PARP1 | F1 | SEQ ID | 32 | (SEQ ID NO: 09) | 479 |
| V762A | | NO: 22 | | TCCCAAATGTCAGCATGTACGA | |
| (rs1136410) | R1 | SEQ ID | 510 | (SEQ ID NO: 10) | |
| | | NO: 22 | | TCCAGGAGATCCTAACACACATGG | |
| | F2 | SEQ ID | 149 | (SEQ ID NO: 11) | 479 |
| | | NO: 22 | | AGGTAACAGGCTGGCCCTGAC | |
| | R2 | SEQ ID | | (SEQ ID NO: 12) | |
| | | NO: 22 | | AGGAAGGCCTGACCCTGTTACC | |

Confidence intervals for the odds ratios of the distributions of individual polymorphisms relative to the wild type between controls and patients with cancer were determined using the exact method. The probability of survival as a function of time since diagnosis was determined by the Kaplan-Meier method. The statistical significance of the differences in survival among the genotypes was determined by the log-rank test. An adjustment was made to the p-value comparing survival among patients with different haplotypes when the grouping was made after examining the data and selecting the better of the possible combinations. Except as noted, all p-values are two-tailed and reported without adjustment for multiple comparisons.

Five hundred and thirteen patients with castrate-resistant prostate cancer were assayed for 5 single nucleotide polymorphisms (SNPs): ERCC1 N118N (C>T), XPD K751Q (A>C), XRCC1 R399Q (G>A), XRCC1 R194W (C>T), and PARP1 V762A (T>C). The distribution of these SNPs among the 513 patients studied was compared to the 152 healthy volunteer controls. Table 3 shows the distribution of polymorphisms among controls and patients. Statistical analyses of the genotype prevalence for all five polymorphisms revealed no evidence of any differences between the two groups. The column of Table 3 entitled ‘Genotype’ provides the identity of the polymorphic nucleotides at each of the alleles in the genome. For example, the genotype CC in the XRCC1 R194W row means that the polymorphic nucleotide in each of the two alleles encoding amino acid 196 of the XRCC1 polypeptide was C. All of the genotype distributions were in Hardy-Weinberg equilibrium in both cases and controls.

TABLE 3

| | | | | | 95% | |
|---|---|---|---|---|---|---|
| | | | | | (Exact | |
| | | Control | Patients | | Confidence | |
| SNP | Genotype | (Number (%)) | (Number (%)) | Odds Ratio | Interval) | P Value |
| ERCC1 | CC | 23 (21) | 91 (21) | Referent | — | — |
| N118N | CT | 53 (49) | 197 (46) | 0.940 | 0.5426 to | 0.8899 |
| (rs11615) | | | | | 1.627 | |
| | TT | 32 (30) | 143 (33) | 1.129 | 0.6218 to | 0.7595 |
| | | | | | 2.052 | |
| XPD | AA | 49 (42) | 186 (43) | Referent | — | — |
| K751Q | AC | 56 (47) | 178 (42) | 0.837 | 0.5419 to | 0.4399 |
| (rs13181) | | | | | 1.294 | |
| | CC | 13 (11) | 64 (15) | 1.297 | 0.6608 to | 0.5129 |
| | | | | | 2.546 | |
| XRCC1 | CC | 120 (87) | 402 (89) | Referent | — | — |
| R194W | CT | 17 (12) | 43 (09) | 0.755 | 0.4154 to | 0.3399 |
| (rs1799782) | | | | | 1.372 | |
| | TT | 1 (01) | 7 (02) | 2.090 | 0.2544 to | 0.6893 |
| | | | | | 17.16 | |
| XRCC1 | GG | 49 (46) | 145 (41) | Referent | — | — |
| R399Q | AG | 47 (44) | 151 (43) | 1.086 | 0.6850 to | 0.8144 |
| (rs25487) | | | | | 1.721 | |
| | AA | 10 (10) | 56 (16) | 1.892 | 0.8967 to | 0.1248 |
| | | | | | 3.994 | |
| PARP1 | TT | 80 (67) | 315 (0.70) | Referent | — | — |
| V762A | CT | 32 (27) | 123 (0.27) | 0.976 | 0.6163 to | 0.9068 |
| (rs1136410) | | | | | 1.546 | |
| | CC | 7 (06) | 15 (0.03) | 0.544 | 0.2147 to | 0.1873 |
| | | | | | 1.380 | |

The univariate method was used to determine whether polymorphisms were associated with overall survival. None of the polymorphisms evaluated showed a trend toward an association with survival individually. Table 4 shows the results including median survival, and two-tailed log-rank test p-values.

TABLE 4

| | | Median | Median survival | Median survival |
|---|---|---|---|---|
| | | survival | for radiation | for non-radiation |
| SNP | Genotype | (years) | group (years) | group (years) |
| ERCC1 | CC | 8.21 | 9.72 | 6.915 |
| N118N | CT | 7.84 | 10.35 | 4.781 |
| (rs11615) | TT | 8.33 | 8.86 | 6.381 |
| | P Value | 0.7622 | 0.9649 | 0.4028 |
| XPD | AA | 8.13 | 8.86 | 6.7 |
| K751Q | AC | 8.21 | 10.33 | 5.32 |
| (rs13181) | CC | 7.155 | 9.22 | 4.15 |
| | P Value | 0.9925 | 0.9325 | 0.6019 |
| XRCC1 | GG | 8.17 | 9.22 | 5.88 |
| R399Q | AG | 7.77 | 10.41 | 5.41 |
| (rs25487) | AA | 11.12 | 11.75 | 8.305 |
| | P Value | 0.5256 | 0.8456 | 0.6261 |
| XRCC1 | CC | 8.06 | 9.66 | 5.88 |
| R194W | CT | 6.52 | 6.81 | 4.24 |
| (rs1799782) | TT | 9.22 | 9.22 | 10.595 |
| | P Value | 0.5493 | 0.3361 | 0.8515 |
| PARP1 | TT | 8.17 | 9.55 | 5.9 |
| V762A | CT | 7.69 | 8.82 | 4.985 |
| (rs1136410) | CC | 5.88 | 11.675 | 3.9 |
| | P Value | 0.8469 | 0.6805 | 0.0949 |

The group of patients having the XRCC1 R399Q (AA) genotype had the longest individual median survival time (11.12 years). The group of patients having the XRCC1 R194W (CT) genotype had the shortest median survival time (6.52 years). Interestingly, patients who received radiotherapy treatment with the XRCC1 R399Q (AA) or XRCC1 R399Q (AG) genotype had median survivals greater than 10 years. In contrast, patients who received radiotherapy treatment with the XRCC1 R194W (CT) genotype had a median survival of 6.81 years. The intragenic association of XRCC1 genotypes with increased overall survival was investigated, including, the R399Q (AA) or (AG) genotypes, and the R194W (CT) genotype. Four haplotypes were found to be associated: R399Q (AA)/R194W (CC); R399Q (AG)/R194W (CC); R399Q (AG)/R914W (CT); and R399Q (GG)/R194W (CT). The XRCC1 R399Q (AA) and the XRCC1 R194W (CT) genotypes showed a tendency to be mutually exclusive. However, a patient displayed the XRCC1 R399Q (AA)/XRCC1 R194W (CT) haplotype, and this patient continued to survive.

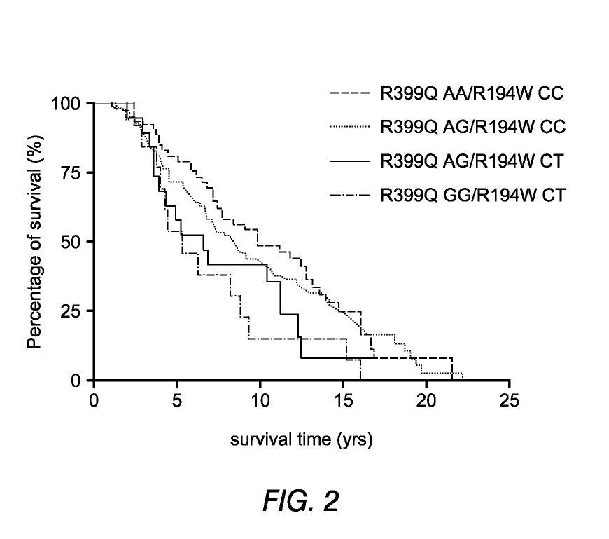

Kaplan-Meier curves for the overall survival of patients with castrate-resistant prostate cancer were plotted (FIG. 2). Each curve represented patients with one of four haplotypes: XRCC1 R399Q AA/XRCC1 R194W CC; XRCC1 R399Q AG/XRCC1 R194W CC; XRCC1 R399Q AG/XRCC1 R194W CT; and XRCC1 R399Q GG/XRCC1 R194W CT. The duration of survival was computed from the date of prostate cancer diagnosis until the date of death or last follow-up. P values were adjusted for haplotype analysis. The median survival time 9.81 years for R399Q AA/R194W CC (n=53), 8.39 years for R399Q AG/R194W CC (n=124), 6.52 years for R399Q AG/R194W CT (n=19) and 5.26 years for R399Q GG/R194W CT (n=13). The global two-tailed p-value=0.14.

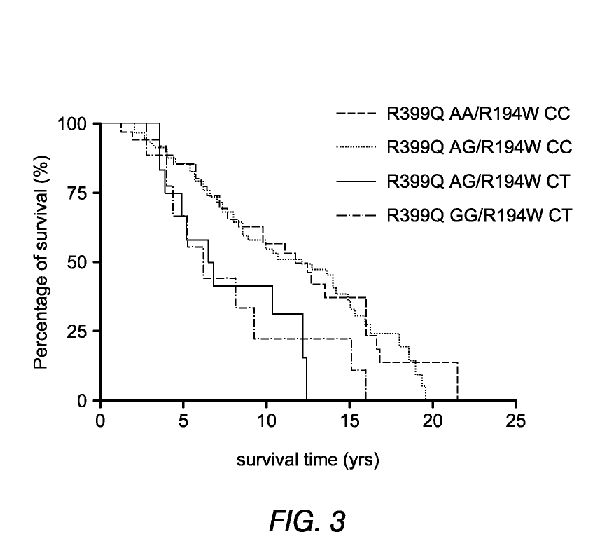

Kaplan-Meier curves for the overall survival of patients with castrate-resistant prostate cancer treated with radiotherapy were plotted (FIG. 3). Each curve represented patients with one of four haplotypes: XRCC1 R399Q AA/XRCC1 R194W CC; XRCC1 R399Q AG/XRCC1 R194W CC; XRCC1 R399Q AG/XRCC1 R194W CT; and XRCC1 R399Q GG/XRCC1 R194W CT. The duration of survival was computed from the date of prostate cancer diagnosis until the date of death or last follow-up. P values were adjusted for haplotype analysis. The median survival time was 11.75 years for R399Q AA/R194W CC (n=35), 12.17 years for R399Q AG/R194W CC genotype (n=63), 6.665 years for R399Q AG/R194W CT (n=12) and 6.21 years for R399Q GG/R194W CT (n=9). The global two-tailed p-value=0.034.

A comparison between the Kaplan-Meier curves for all patients with castrate-resistant prostate cancer (FIG. 2), and all patents with castrate-resistant prostate cancer treated with radiotherapy (FIG. 3) suggests that XRCC1 is a prognostic marker for radiotherapy in prostate cancer.

In the NCI-60 cell line screening experiment, the genotypes of the 5 SNPs: ERCC1 N118N (500C>T), XPD K751Q (2282A>C), XRCC1 R399Q (1301G>A), XRCC1 R194W (685C>T), and PARP1 V762A (2446T>C), did not show significant correlation to the sensitivity to DNA damaging chemotherapy agents cisplatin, carboplatin, oxaliplatin, and tetraplatin as reported previously (Rixe O et al. Oxaliplatin, tetraplatin, cisplatin, and carboplatin: spectrum of activity in drug-resistant cell lines and in the cell lines of the National Cancer Institute's Anticancer Drug Screen panel. Biochem Pharmacol. 1996 Dec. 24: 52:1855-65).

Several patterns were observed in the data. First, all five SNPs assessed in this study were not associated with prostate cancer as compared to healthy volunteers. Second, there was a significant trend in patient survival to suggest the possibility that the XRCC1 R399Q genotype in combination with the XRCC1 R194W may have an impact on the outcome of radiotherapy in prostate cancer. Neither the XRCC1 R399Q nor the XRCC1 R194W was associated with overall survival individually (p=0.5256 and 0.5493, respectively). However, the combination of R399Q and R194W genotypes showed correlation to the overall survival in the patients receiving radiotherapy in prostate cancer. Patients possessing at least one variant allele A of R399Q and wild type CC of R194W had significantly longer survival time after radiotherapy, while patients having at least one wild type allele G of R399Q and the heterozygous genotype CT of R194W had shorter survival time (p=0.034). This outcome was not observed when patients received therapies other than radiation were included.

The genotype of XRCC1 R399Q is a prognostic factor to radiation therapy in patients with prostate cancer, and this effect is modified by the R194W genotype.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

What is claimed is:

1.-88. (canceled)

89. A method for evaluating a prognosis of a subject with a prostate neoplastic condition comprising: determining the genotype of said subject at least one codon selected from the group consisting of the codon encoding amino acid 399 of the XRCC1 polypeptide, the codon encoding amino acid 194 of the XRCC1 polypeptide, and the codon encoding amino acid 762 of the PARP1 polypeptide.

90. The method of claim 89, wherein said step of determining the genotype comprises determining the identity of a polymorphic nucleotide selected from the group consisting of the polymorphic nucleotide at position 1316 of SEQ ID NO:17 or a polymorphic nucleotide corresponding thereto, the polymorphic nucleotide at position 700 of SEQ ID NO:17 or a polymorphic nucleotide corresponding thereto, and the polymorphic nucleotide at position 2456 of SEQ ID NO:19 or a polymorphic nucleotide corresponding thereto.

91. The method of claim 90, wherein said determining step the genotype comprises a step selected from the group consisting of extending a primer that hybridizes to a sequence adjacent to the polymorphic nucleotide, and hybridizing a probe to a region that includes the polymorphic nucleotide.

92. The method of claim 89, further comprising obtaining a sample from said subject.

93. The method of claim 89, further comprising providing the result of said determining step to a party in order for said party to select a treatment for said prostate neoplastic condition in said subject.

94. The method of claim 90, wherein said genotype is at least one genotype selected from the group consisting of XRCC1 R399Q AA, PARPI V762A CC, XRCC1 R194W CC, XRCC1 R399Q AG, XRCC1 R194W CT, and XRCC1 R399Q GG.

95. The method of claim 90, wherein said genotype is at least one genotype selected from the group consisting of AA for the polymorphic nucleotide at position 1316 of SEQ ID NO:17 or a polymorphic nucleotide corresponding thereto, CC for the polymorphic nucleotide at position 2456 of SEQ ID NO:19, CC for the polymorphic nucleotide at position 700 of SEQ ID NO:17 or a polymorphic nucleotide corresponding thereto, AG for the polymorphic nucleotide at position 1316 of SEQ ID NO:17 or a polymorphic nucleotide corresponding thereto, CT for the polymorphic nucleotide at position 700 of SEQ ID NO:17 or a polymorphic nucleotide corresponding thereto, and GG for the polymorphic nucleotide at position 1316 of SEQ ID NO:17 or a polymorphic nucleotide corresponding thereto.

96. The method of claim 90, wherein the presence of at least one genotype selected from the group consisting of XRCC1 R399Q AA, PARP1 V762A CC, and XRCC1 R194W CC indicates a favorable prognosis.

97. The method of claim 90, wherein the presence of at least one genotype selected from the group consisting of AA for the polymorphic nucleotide at position 1316 of SEQ ID NO:17 or a polymorphic nucleotide corresponding thereto, CC for the polymorphic nucleotide at position 2456 of SEQ ID NO:19 or a polymorphic nucleotide corresponding thereto, and CC for the polymorphic nucleotide at position 700 of SEQ ID NO:17 or a polymorphic nucleotide corresponding thereto indicates a favorable prognosis.

98. The method of claim 90, wherein the presence of CC for the polymorphic nucleotide at position 700 of SEQ ID NO:17 or a polymorphic nucleotide corresponding thereto and AA for the polymorphic nucleotide at position 1316 of SEQ ID NO:17 or a polymorphic nucleotide corresponding thereto together, or CC for the polymorphic nucleotide at position 700 of SEQ ID NO:17 or a polymorphic nucleotide corresponding thereto and AG for the polymorphic nucleotide at position 1316 of SEQ ID NO:17 or a polymorphic nucleotide corresponding thereto together indicates a favorable prognosis.

99. The method of claim 90, wherein the presence of at least one genotype selected from the group consisting of XRCC1 R194W CT and XRCC1 R399Q GG indicates an unfavorable prognosis.

100. The method of claim 90, wherein the presence of at least one genotype selected from the group consisting of CT for the polymorphic nucleotide at position 700 of SEQ ID NO:17 or a polymorphic nucleotide corresponding thereto and GG for the polymorphic nucleotide at position 1316 of SEQ ID NO:17 or a polymorphic nucleotide corresponding thereto indicates an unfavorable prognosis.

101. The method of claim 90, wherein the presence of XRCC1 R194W CT, and XRCC1 R399Q AG, or XRCC1 R399Q GG indicates an unfavorable prognosis.

102. The method of claim 90, wherein the presence of CT for the polymorphic nucleotide at position 700 of SEQ ID NO:17 or a polymorphic nucleotide corresponding thereto, and AG for the polymorphic nucleotide at position 1316 of SEQ ID NO:17 or a polymorphic nucleotide corresponding thereto, or GG for the polymorphic nucleotide at position 1316 of SEQ ID NO:17 or a polymorphic nucleotide corresponding thereto indicates an unfavorable prognosis.

103. The method of claim 89, wherein said prognosis comprises a favorable or unfavorable response to radiation therapy.

104. The method of claim 89, wherein a favorable prognosis comprises a period for overall survival for said subject which is at least 1 year greater than the period of overall survival for a subject with an unfavorable prognosis.

105. The method of claim 89, further comprising administering a treatment for which the determined genotype is indicative of a favorable response.

106. The method of claim 89, wherein said condition is castrate-resistant prostate cancer.

107. A method for evaluating the response to radiation therapy in a subject with a prostate neoplastic condition comprising:
determining the genotype of said subject at at least one codon selected from the group consisting of the codon encoding amino acid 399 of the XRCC1 polypeptide, the codon encoding amino acid 194 of the XRCC1 polypeptide, and the codon encoding amino acid 762 of the PARP1 polypeptide; and
providing the result of said evaluating to a party in order for said party to select a treatment for said subject.

108. The method of claim 107, wherein said step of determining, the genotype comprises determining the identity of a polymorphic nucleotide selected from the group consisting of the polymorphic nucleotide at position 1316 of SEQ ID NO:17 or a polymorphic nucleotide corresponding thereto, the polymorphic nucleotide at position 700 of SEQ ID NO:17, and the polymorphic nucleotide at position 2456 of SEQ ID NO:19 or a polymorphic nucleotide corresponding thereto.

109. The method of claim 108, wherein said determining the genotype comprises a step selected from the group consisting of extending a primer that hybridizes to a sequence adjacent to the polymorphic nucleotide, and hybridizing a probe to a region that includes the polymorphic nucleotide.

110. The method of claim 107, further comprising obtaining a sample from said subject.

111. The method of claim 108, wherein said genotype is at least one genotype selected from the group consisting of XRCC1 R399Q AA, PARPI V762A CC, XRCC1 R194W CC, XRCC1 R399Q AG, XRCC1 R194W CT, and XRCC1 R399Q GG.

112. The method of claim 108, wherein said genotype is at least one genotype selected from the group consisting of AA for the polymorphic nucleotide at position 1316 of SEQ ID NO:17 or a polymorphic nucleotide corresponding thereto, CC for the polymorphic nucleotide at position 2456 of SEQ ID NO:19, CC for the polymorphic nucleotide at position 700 of SEQ ID NO:17 or a polymorphic nucleotide corresponding thereto, AG for the polymorphic nucleotide at position 1316 of SEQ ID NO:17 or a polymorphic nucleotide corresponding thereto, CT for the polymorphic nucleotide at position 700 of SEQ ID NO:17 or a polymorphic nucleotide corresponding thereto, and GG for the polymorphic nucleotide at position 1316 of SEQ ID NO:17 or a polymorphic nucleotide corresponding thereto.

113. The method of claim 108, wherein the presence of at least one genotype selected from the group consisting of XRCC1 R399Q AA, PARP1 V762A CC, and XRCC1 R194W CC indicates a favorable prognosis.

114. The method of claim 108, wherein the presence of at least one genotype selected from the group consisting of AA for the polymorphic nucleotide at position 1316 of SEQ ID NO:17 or a polymorphic nucleotide corresponding thereto, CC for the polymorphic nucleotide at position 2456 of SEQ ID NO:19 or a polymorphic nucleotide corresponding thereto, and CC for the polymorphic nucleotide at position 700 of SEQ ID NO:17 or a polymorphic nucleotide corresponding thereto indicates a favorable prognosis.

115. The method of claim 108, wherein the presence of CC for the polymorphic nucleotide at position 700 of SEQ ID NO:17 or a polymorphic nucleotide corresponding thereto and AA for the polymorphic nucleotide at position 1316 of SEQ ID NO:17 or a polymorphic nucleotide corresponding thereto together, or CC for the polymorphic nucleotide at position 700 of SEQ ID NO:17 or a polymorphic nucleotide corresponding thereto and AG for the polymorphic nucleotide at position 1316 of SEQ ID NO:17 or a polymorphic nucleotide corresponding thereto together indicates a favorable prognosis.

116. The method of claim 108, wherein the presence of at least one genotype selected from the group consisting of XRCC1 R194W CT and XRCC1 R399Q GG indicates an unfavorable prognosis.

117. The method of claim 108, wherein the presence of at least one genotype selected from the group consisting of CT for the polymorphic nucleotide at position 700 of SEQ ID NO:17 or a polymorphic nucleotide corresponding thereto and GG for the polymorphic nucleotide at position 1316 of SEQ ID NO:17 or a polymorphic nucleotide corresponding thereto indicates an unfavorable prognosis.

118. The method of claim 108, wherein the presence of XRCC1 R194W CT, and XRCC1 R399Q AG, or XRCC1 R399Q GG indicates an unfavorable prognosis.

119. The method of claim 108, wherein the presence of CT for the polymorphic nucleotide at position 700 of SEQ ID NO:17 or a polymorphic nucleotide corresponding thereto, and AG for the polymorphic nucleotide at position 1316 of SEQ ID NO:17 or a polymorphic nucleotide corresponding thereto, or GG for the polymorphic nucleotide at position 1316 of SEQ ID NO:17 or a polymorphic nucleotide corresponding thereto indicates an unfavorable prognosis

120. The method of claim 107, wherein a favorable prognosis comprises an overall survival at least 1 year greater than the overall survival of an unfavorable prognosis.

121. The method of claim 107, wherein said condition is castrate-resistant prostate cancer.

122. A method for selecting a treatment for a subject with a prostate neoplastic condition comprising:
determining the genotype of said subject at at least one codon selected from the group consisting of the codon encoding amino acid 399 of the XRCC1 polypeptide, the codon encoding amino acid 194 of the XRCC1 polypeptide, and the codon encoding amino acid 762 of the PARP1 polypeptide; and
selecting a treatment for said subject based on the determined genotype.

123. The method of claim 122, wherein said step at determining the genotype comprises determining the identity of a polymorphic nucleotide selected from the group consisting the polymorphic nucleotide at position 1316 of SEQ ID NO:17 or a polymorphic nucleotide corresponding thereto, the polymorphic nucleotide at position 700 of SEQ ID NO:17 or a polymorphic nucleotide corresponding thereto, and the polymorphic nucleotide at position 2456 of SEQ ID NO:19 or a polymorphic nucleotide corresponding thereto.

124. The method of claim 123, wherein said determining the genotype comprises a step selected from the group consisting of extending a primer that hybridizes to a sequence adjacent to the polymorphic nucleotide, and hybridizing a probe to a region that includes the polymorphic nucleotide.

125. The method of claim 122, further comprising obtaining a sample from said subject.

126. The method of claim 123, wherein said genotype is at least one genotype selected from the group consisting of XRCC1 R399Q AA, PARPI V762A CC, XRCC1 R194W CC, XRCC1 R399Q AG, XRCC1 R194W CT, and XRCC1 R399Q GG.

127. The method of claim 123, wherein said genotype is at least one genotype selected from the group consisting of AA for the polymorphic nucleotide at position 1316 of SEQ ID NO:17 or a polymorphic nucleotide corresponding thereto, CC for the polymorphic nucleotide at position 2456 of SEQ ID NO:19, CC for the polymorphic nucleotide at position 700 of SEQ ID NO:17 or a polymorphic nucleotide corresponding thereto, AG for the polymorphic nucleotide at position 1316 of SEQ ID NO:17 or a polymorphic nucleotide corresponding thereto, CT for the polymorphic nucleotide at position 700 of SEQ ID NO:17 or a polymorphic nucleotide corresponding thereto, and GG for the polymorphic nucleotide at position 1316 of SEQ ID NO:17 or a polymorphic nucleotide corresponding thereto.

128. The method of claim 123, wherein the presence of at least one genotype selected from the group consisting of XRCC1 R399Q AA, PARP1 V762A CC, and XRCC1 R194W CC indicates a favorable prognosis.

129. The method of claim 123, wherein the presence of at least one genotype selected from the group consisting of AA for the polymorphic nucleotide at position 1316 of SEQ ID NO:17 or a polymorphic nucleotide corresponding thereto, CC for the polymorphic nucleotide at position 2456 of SEQ ID NO:19 or a polymorphic nucleotide corresponding thereto, and CC for the polymorphic nucleotide at position 700 of SEQ ID NO:17 or a polymorphic nucleotide corresponding thereto indicates a favorable prognosis.

130. The method of claim 123, wherein the presence of CC for the polymorphic nucleotide at position 700 of SEQ ID NO:17 or a polymorphic nucleotide corresponding thereto and AA for the polymorphic nucleotide at position 1316 of SEQ ID NO:17 or a polymorphic nucleotide corresponding thereto together, or CC for the polymorphic nucleotide at position 700 of SEQ ID NO:17 or a polymorphic nucleotide corresponding thereto and AG for the polymorphic nucleotide at position 1316 of SEQ ID NO:17 or a polymorphic nucleotide corresponding thereto together indicates a favorable prognosis.

131. The method of claim 123, wherein the presence of at least one genotype selected from the group consisting of XRCC1 R194W CT and XRCC1 R399Q GG indicates an unfavorable prognosis.

132. The method of claim 123, wherein the presence of at least one genotype selected from the group consisting of CT for the polymorphic nucleotide at position 700 of SEQ ID NO:17 or a polymorphic nucleotide corresponding thereto and GG for the polymorphic nucleotide at position 1316 of SEQ ID NO:17 or a polymorphic nucleotide corresponding thereto indicates an unfavorable prognosis.

133. The method of claim 123, wherein the presence of XRCC1 R194W CT, and XRCC1 R399Q AG, or XRCC1 R399Q GG indicates an unfavorable prognosis.

134. The method of claim 123, wherein the presence of CT for the polymorphic nucleotide at position 700 of SEQ ID NO:17 or a polymorphic nucleotide corresponding thereto, and AG for the polymorphic nucleotide at position 1316 of SEQ ID NO:17 or a polymorphic nucleotide corresponding thereto, or GG for the polymorphic nucleotide at position 1316 of SEQ ID NO:17 or a polymorphic nucleotide corresponding thereto indicates an unfavorable prognosis.

135. The method of claim 122, wherein a favorable prognosis comprises an overall survival at least 1 year greater than the overall survival of an unfavorable prognosis.

136. The method of claim 122, wherein said condition is castrate-resistant prostate cancer.

137. A kit for evaluating a response to radiation therapy in a subject with a prostate neoplastic condition comprising:
a primer or probe which can be used to identify a genotype of the codon encoding amino acid 339 of the XRCC1 polypeptide; and
a primer or probe which can be used to identify the genotype of the codon encoding amino acid 194 of the XRCC1 polypeptide.

138. The kit of claim 137, wherein said primer or probe which can be used to identify a genotype of the codon encoding amino acid 339 of the XRCC1 polypeptide can be used to identify a polymorphic nucleotide at position 1316 of SEQ ID NO:17 or a polymorphic nucleotide corresponding thereto, and said primer or probe which can be used to identify a genotype of the codon encoding amino acid 194 of the XRCC1 polypeptide can be used to identify a polymorphic nucleotide at position 700 of SEQ ID NO:17 or a polymorphic nucleotide corresponding thereto.

139. The kit of claim 137, further comprising a primer or probe which can be used to identify the genotype of the codon encoding amino acid 762 of the PARP1 polypeptide.

140. The kit of claim 137, wherein said primer or probe which can be used to identify a genotype of the codon encoding amino acid 762 of the PARP1 polypeptide can be used to identify a polymorphic nucleotide at position 2456 of SEQ ID NO:19 or a polymorphic nucleotide corresponding thereto.

141. A method for identifying one or more polymorphisms in the XRCC1 gene which is associated with a favorable or unfavorable response to radiation therapy in a subject having a prostate neoplastic condition comprising:
determining the identity of one or more polymorphic nucleotides in the XRCC1 gene in a plurality of individuals having a prostate neoplastic condition who responded favorably to radiation therapy;
determining the identity of one or more polymorphic nucleotides in the XRCC1 gene in a plurality of individuals having a prostate neoplastic condition who responded unfavorably to radiation therapy; and
identifying one or more polymorphisms having a statistically significant correlation with a favorable response to radiation therapy.

142. The method of claim 141, wherein said determining is performed in an automated device.

143. A method for identifying one or more polymorphisms in the XRCC1 gene which is associated with a favorable or unfavorable response to radiation therapy in a subject having a prostate neoplastic condition comprising:
determining the identity of one or more polymorphic nucleotides in the XRCC1 gene in a plurality of individuals having a prostate neoplastic condition who responded favorably to radiation therapy;
determining the identity of one or more polymorphic nucleotides in the XRCC1 gene in a plurality of individuals having a prostate neoplastic condition who responded unfavorably to radiation therapy; and
identifying one or more polymorphisms having a statistically significant correlation with a favorable response to radiation therapy.

144. The method of claim 143, wherein said determining is performed in an automated device.

145. A method of treating a subject with a prostate neoplastic condition comprising:
determining the genotype of said subject at at least one codon selected from the group consisting of the codon encoding amino acid 399 of the XRCC1 polypeptide, the codon encoding amino acid 194 of the XRCC1 polypeptide, and the codon encoding amino acid 762 of the PARP1 polypeptide; and
treating said subject with radiation therapy if said subject has at least one genotype selected from the group consisting of XRCC1 R399Q AA, PARP1 V762A CC, and XRCC1 R194W CC.

146. The method of claim 143, wherein said step of determining the genotype comprises determining the identity of a polymorphic nucleotide selected from the group consisting of the polymorphic nucleotide at position 1316 of SEQ ID NO:17 or a polymorphic nucleotide corresponding thereto, the polymorphic nucleotide at position 700 of SEQ ID NO:17 or a polymorphic nucleotide corresponding thereto, and the polymorphic nucleotide at position 2456 of SEQ ID NO:19 or a polymorphic nucleotide corresponding thereto.

147. The method of claim 143, wherein said radiation therapy is selected from external beam radiotherapy and brachytherapy.

148. The method of claim 143, wherein said condition is castrate-resistant prostate cancer.

149. A method of treating a subject with a prostate neoplastic condition comprising:
determining the genotype of said subject at at least one codon selected from the group consisting of the codon encoding amino acid 399 of the XRCC1 polypeptide, the codon encoding amino acid 194 of the XRCC1 polypeptide, and the codon encoding amino acid 762 of the PARP1 polypeptide; and
treating said subject with a treatment selected from the group consisting of surgery, chemotherapy, cryosurgery, and high intensity focused ultrasound if said subject has at least one genotype selected from the group consisting of XRCC1 R194W CT, XRCC1 R399Q GG, XRCC1 R194W CT and XRCC1 R399Q AG, and XRCC1 R194W CT XRCC1 R399Q.


SEQUENCE LISTING (22 sequences)

SEQ ID NO: 1 - DNA, 20 bp, Homo Sapien
  ERCC1
tggatcagaggatcagggac

SEQ ID NO: 2 - DNA, 20 bp, Homo Sapien
  ERCC1
ttcctgagacccaggagttc

SEQ ID NO: 3 - DNA, 24 bp, Homo Sapien
  XPD
ccttctcctgcgattaaaggctgt

SEQ ID NO: 4 - DNA, 24 bp, Homo Sapien
  XPD
tcagccccatcttatgttgacagg

SEQ ID NO: 5 - DNA, 20 bp, Homo Sapien
  XRCC1
agacaaagatgaggcagagg

SEQ ID NO: 6 - DNA, 21 bp, Homo Sapien
  XRCC1
tcaaccctcaggacacaagag

SEQ ID NO: 7 - DNA, 21 bp, Homo Sapien
  XRCC1
tgcatctctcccttggtctcc

SEQ ID NO: 8 - DNA, 21 bp, Homo Sapien
  XRCC1
tgcacaaactgctcctccagc

SEQ ID NO: 9 - DNA, 22 bp, Homo Sapien
  PARP1
tcccaaatgtcagcatgtacga

SEQ ID NO: 10 - DNA, 24 bp, Homo Sapien
  PARP1
tccaggagatcctaacacacatgg

SEQ ID NO: 11 - DNA, 21 bp, Homo Sapien
  PARP1
aggtaacaggctggccctgac

SEQ ID NO: 12 - DNA, 22 bp, Homo Sapien
  PARP1
aggaaggcctgaccctgttacc

SEQ ID NO: 13 - DNA, 3328 bp, Homo Sapien
  mRNA (1)...(3328): ERCC1
ccggaagtgctgcgagccctgggccacgctggccgtgctggcagtgggccgcctcgatccctctgcagtctttcccttgaggctccaagaccagcaggtgaggcctcgcggcgctgaaaccgtgaggcccggaccacaggctccagatggaccctgggaaggacaaagagggggtgccccagccctcagggccgccagcaaggaagaaatttgtgatacccctcgacgaggatgaggtccctcctggagtggccaagcccttattccgatctacacagagccttcccactgtggacacctcggcccaggcggcccctcagacctacgccgaatatgccatctcacagcctctggaaggggctggggccacgtgccccacagggtcagagcccctggcaggagagacgcccaaccaggccctgaaacccggggcaaaatccaacagcatcattgtgagccctcggcagaggggcaatcccgtactgaagttcgtgcgcaatgtgccctgggaatttggcgacgtaattcccgactatgtgctgggccagagcacctgtgccctgttcctcagcctccgctaccacaacctgcacccagactacatccatgggcggctgcagagcctggggaagaacttcgccttgcgggtcctgcttgtccaggtggatgtgaaagatccccagcaggccctcaaggagctggctaagatgtgtatcctggccgactgcacattgatcctcgcctggagccccgaggaagctgggcggtacctggagacctacaaggcctatgagcagaaaccagcggacctcctgatggagaagctagagcaggacttcgtctcccggtctctggaacagctcatcgccgcatcaagagaagatctggccttatgcccaggcctgggccctcagaaagcccggaggctgtttgatgtcctgcacgagcccttcttgaaagtaccctgatgaccccagctgccaaggaaacccccagtgtaataataaatcgtcctcccaggccaggctcctgctggctgcgctggtgcagtctctggggagggattctgggggtgtcaccttctggtggcccaggtgggcaccttcagctttctttagttcctcagtttcccgggggcagactacacaggctgctgctgctgctgcttccgcttcttgtcccggcctgtgggagcctcctccccagactctgaattcagtggcggccctggcatctcctcttggggcactgtctctggcatccggctttcctgactctgcttcttcctcttcttggtggatcccggagttgccctggcttcaggctgtccctcccctggcagttcaggctctagtggctgaattggctcagtcactgtgtgacctctctctttcttcttcttcttcttcttggtggatgtgggagctgcctgaggctcaaggtcatccggcagctcaggccccaccacctctgtctctggctccactgtggcatcttgctgtttttctttcttcgtcttctttttgggagctgccagagctgcctgggcctgaggcttcgctccttctggctgttgaggcgccatggtcccccctggggactccagaggcttcatctccggctccactggctccatcgcctccgtccctggctccatcattgccatctgtcccttttcttttttcctcttcttcgtagggggcagagggatggcttcctccagtggctccaccttcacctgtggctgagactcaactgtcaccccctcctctggctccatcccttccgtccccttttgcctctttctctttttggtcggggacaggactgtgtcttctagaggctcagtgttaatctgttcctgcttcactgtcttgtcttctggctcgaaggtttctttccctttgggcttcttcctcttcttggtggtggacgggaacagcactcccagaggctccagtgtctccactgtgggctctgtccccacaggccctgctgcctctggttctttcagctgctgattttttttcttcttcttcttccgcacatccatttctggcgaccccaaagccatgtccacctccagggccccgtgcccattcactgcctcctgagtgactggggcctctgtcacctgcatctcctttttcttcttccctgaggtgagcaggttgggggccaaggctgacctaggccctgtgactggtgggttgcccccaaaggcacagaaccgaggcctcaggccaggagggatctgtggtgggggacttgctgggatgggctgcagagggctccctgacagggattgctggggaccctcaaggatccttagggtgccctggggggctgaggcacaggtgagtccacctcctgcctccgttgagggggccagcagggtcgcttctccagcttggggacagctgctgaggactcgatagcggtgccgcttgcctgccaatttgcccttgacgatctgggagccagagagaggcacatgccgcccattgaagctacagagagaaacagggagggcagaggcttaagtggaacaggagagggaaggttttttgattttttttttgtttttttttgagagagtcttgctctgttgcctaggctggagtgcagtggcatgatctcggctcactgcaatgtccacctcctgggttcaagcgattctcctgcctcagcctctcaagtagctgggattacaggcacctgccaccacgcccagccaatttttgtatttttagtagagacaatttcactatgttggccaggctggtcttgaactcctgacctcaagtgatctgctcgcctcggcctcccaaaggatgggattacaggcaccagccactgcgcctggctggcctctggtttttaataaaacatgactagagtgactccatcttaaagtgagtagctaggcacttacaaggttcatgcttatggcctgaaaataaccacatcccaggctgaccaccaattataattacagaatatttatggccatacagaacatgttccaccaagcctgcagaatgtccaaatgtcctaagaatgcagcccccattacttaaatataacataaatgagcaagcttaggttgcaggattaatggtcgtggataacaccaatagcccctacctttagtgagcttatctgcacactccaagtttaactatagttccttatagtttcttataagtagaaatactaacaaagggctgtgggtttctccccctgctttctgaggacactctactctgtaaaggagtagtttccaataaacttgtttctttcactgtgcaaaaaaaaaaaaaaaaaa

SEQ ID NO: 14 - protein, 273 aa, Homo Sapien
  PEPTIDE (1)...(270): ERCC1
MDPGKDKEGVPQPSGPPARKKFVIPLDEDEVPPGVAKPLFRSTQSLPTVDTSAQAAPQTYAEYAISQPLEGAGATCPTGSEPLAGETPNQALKPGAKSNSIIVSPRQRGNPVLKFVRNVPWEFGDVIPDYVLGQSTCALFLSLRYHNLHPDYIHGRLQSLGKNFALRVLLVQVDVKDPQQALKELAKMCILADCTLILAWSPEEAGRYLETYKAYEQKPADLLMEKLEQDFVSRSLEQLIAASREDLALCPGLGPQKARRLFDVLHEPFLKVP

SEQ ID NO: 15 - DNA, 2568 bp, Homo Sapien
  mRNA (1)...(2568): XPD
ttcatgagggaggcgggtcgaccccgctgcacagtccggccggcgccatgaagctcaacgtggacgggctcctggtctacttcccgtacgactacatctaccccgagcagttctcctacatgcgggagctcaaacgcacgctggacgccaagggtcatggagtcctggagatgccctcaggcaccgggaagacagtatccctgttggccctgatcatggcataccagagagcatatccgctggaggtgaccaaactcatctactgctcaagaactgtgccagagattgagaaggtgattgaagagcttcgaaagttgctcaacttctatgagaagcaggagggcgagaagctgccgtttctgggactggctctgagctcccgcaaaaacttgtgtattcaccctgaggtgacacccctgcgctttgggaaggacgtcgatgggaaatgccacagcctcacagcctcctatgtgcgggcgcagtaccagcatgacaccagcctgccccactgccgattctatgaggaatttgatgcccatgggcgtgaggtgcccctccccgctggcatctacaacctggatgacctgaaggccctggggcggcgccagggctggtgcccatacttccttgctcgatactcaatcctgcatgccaatgtggtggtttatagctaccactacctcctggaccccaagattgcagacctggtgtccaaggaactggcccgcaaggccgtcgtggtcttcgacgaggcccacaacattgacaacgtctgcatcgactccatgagcgtcaacctcacccgccggacccttgaccggtgccagggcaacctggagaccctgcagaagacggtgctcaggatcaaagagacagacgagcagcgcctgcgggacgagtaccggcgtctggtggaggggctgcgggaggccagcgccgcccgggagacggacgcccacctggccaaccccgtgctgcccgacgaagtgctgcaggaggcagtgcctggctccatccgcacggccgagcatttcctgggcttcctgaggcggctgctggagtacgtgaagtggcggctgcgtgtgcagcatgtggtgcaggagagcccgcccgccttcctgagcggcctggcccagcgcgtgtgcatccagcgcaagcccctcagattctgtgctgaacgcctccggtccctgctgcatactctggagatcaccgaccttgctgacttctccccgctcaccctccttgctaactttgccacccttgtcagcacctacgccaaaggcttcaccatcatcatcgagccctttgacgacagaaccccgaccattgccaaccccatcctgcacttcagctgcatggacgcctcgctggccatcaaacccgtatttgagcgtttccagtctgtcatcatcacatctgggacactgtccccgctggacatctaccccaagatcctggacttccaccccgtcaccatggcaaccttcaccatgacgctggcacgggtctgcctctgccctatgatcatcggccgtggcaatgaccaggtggccatcagctccaaatttgagacccgggaggatattgctgtgatccggaactatgggaacctcctgctggagatgtccgctgtggtccctgatggcatcgtggccttcttcaccagctaccagtacatggagagcaccgtggcctcctggtatgagcaggggatccttgagaacatccagaggaacaagctgctctttattgagacccaggatggtgccgaaaccagtgtcgccctggagaagtaccaggaggcctgcgagaatggccgcggggccatcctgctgtcagtggcccggggcaaagtgtccgagggaatcgactttgtgcaccactacgggcgggccgtcatcatgtttggcgtcccctacgtctacacacagagccgcattctcaaggcgcggctggaatacctgcgggaccagttccagattcgtgagaatgactttcttaccttcgatgccatgcgccacgcggcccagtgtgtgggtcgggccatcaggggcaagacggactacggcctcatggtctttgccgacaagcggtttgcccgtggggacaagcgggggaagctgccccgctggatccaggagcacctcacagatgccaacctcaacctgaccgtggacgagggtgtccaggtggccaagtacttcctgcggcagatggcacagcccttccaccgggaggatcagctgggcctgtccctgctcagcctggagcagctagaatcagaggagacgctgaagaggatagagcagattgctcagcagctctgagtggggcgggtggggccataaacggttcctggtgactcctgagtcttgcctggccctggttcccagcggcggtggtgctagaaggtcttatgaagtcaggtgacatttctcactgtcacgtccacagcctttaatcgcaggagaaggcagctatccaccaggtacccagaggcaagggggggccaggagatgatagaccccctctcaccccaccagcccatccctcctgcactgttcc

SEQ ID NO: 16 - protein, 760 aa, Homo Sapien
  PEPTIDE (1)...(760): XPD
MKLNVDGLLVYFPYDYIYPEQFSYMRELKRTLDAKGHGVLEMPSGTGKTVSLLALIMAYQRAYPLEVTKLIYCSRTVPEIEKVIEELRKLLNFYEKQEGEKLPFLGLALSSRKNLCIHPEVTPLRFGKDVDGKCHSLTASYVRAQYQHDTSLPHCRFYEEFDAHGREVPLPAGIYNLDDLKALGRRQGWCPYFLARYSILHANVVVYSYHYLLDPKIADLVSKELARKAVVVFDEAHNIDNVCIDSMSVNLTRRTLDRCQGNLETLQKTVLRIKETDEQRLRDEYRRLVEGLREASAARETDAHLANPVLPDEVLQEAVPGSIRTAEHFLGFLRRLLEYVKWRLRVQHVVQESPPAFLSGLAQRVCIQRKPLRFCAERLRSLLHTLEITDLADFSPLTLLANFATLVSTYAKGFTIIIEPFDDRTPTIANPILHFSCMDASLAIKPVFERFQSVIITSGTLSPLDIYPKILDFHPVTMATFTMTLARVCLCPMIIGRGNDQVAISSKFETREDIAVIRNYGNLLLEMSAVVPDGIVAFFTSYQYMESTVASWYEQGILENIQRNKLLFIETQDGAETSVALEKYQEACENGRGAILLSVARGKVSEGIDFVHHYGRAVIMFGVPYVYTQSRILKARLEYLRDQFQIRENDFLTFDAMRHAAQCVGRAIRGKTDYGLMVFADKRFARGDKRGKLPRWIQEHLTDANLNLTVDEGVQVAKYFLRQMAQPFHREDQLGLSLLSLEQLESEETLKRIEQIAQQL

SEQ ID NO: 17 - DNA, 2102 bp, Homo Sapien
  XRCC1
ctcgcgcgcttgcgcactttagccagcgcagggcgcaccccgccccctcccactctccctgcccctcggaccccatactctacctcatccttctggccaggcgaagcccacgacgttgacatgccggagatccgcctccgccatgtcgtgtcctgcagcagccaggactcgactcactgtgcagaaaatcttctcaaggcagacacttaccgaaaatggcgggcagccaaggcaggcgagaagaccatctctgtggtcctacagttggagaaggaggagcagatacacagtgtggacattgggaatgatggctcagctttcgtggaggtgctggtgggcagttcagctggaggcgctggggagcaagactatgaggtccttctggtcacctcatctttcatgtccccttccgagagccgcagtggctcaaaccccaaccgcgttcgcatgtttgggcctgacaagctggtccgggcagccgccgagaagcgctgggaccgggtcaaaattgtttgcagccagccctacagcaaggactccccctttggcttgagttttgtacggtttcatagccccccagacaaagatgaggcagaggccccgtcccagaaggtgacagtgaccaagcttggccagttccgtgtgaaggaggaggatgagagcgccaactctctgaggccgggggctctcttcttcagccggatcaacaagacatccccagtcacagccagcgacccagcaggacctagctatgcagctgctaccctccaggcttctagtgctgcctcctcagcctctccagtctccagggccataggcagcacctccaagccccaggagtctcccaaagggaagaggaagttggatttgaaccaagaagaaaagaagacccccagcaaaccaccagcccagctgtcgccatctgttcccaagagacctaaattgccagctccaactcgtaccccagccacagccccagtccctgcccgagcacagggggcagtgacaggcaaaccccgaggagaaggcaccgagcccagacgaccccgagctggcccagaggagctggggaagatccttcagggtgtggtagtggtgctgagtggcttccagaaccccttccgctccgagctgcgagataaggccctagagcttggggccaagtatcggccagactggacccgggacagcacgcacctcatctgtgcctttgccaacacccccaagtacagccaggtcctaggcctgggaggccgcatcgtgcgtaaggagtgggtgctggactgtcaccgcatgcgtcggcggctgccctcccagaggtacctcatggcagggccaggttccagcagtgaggaggatgaggcctctcacagcggtggcagcggagatgaagcccccaagcttcctcagaagcaaccccagaccaaaaccaagcccactcaggcagctggacccagctcaccccagaagcccccaacccctgaagagaccaaagcagcctcaccagtgctccaggaagatatagacattgagggggtacagtcagaaggacaggacaatggggcggaagattctggggacacagaggatgagctgaggagggtggcagagcagaaggaacacagactgccccctggccaggaggagaatggggaagacccgtatgcaggctccacggatgagaacacggacagtgaggaacaccaggagcctcctgatctgccagtccctgagctcccagatttcttccagggcaagcacttctttctttacggggagttccctggggacgagcggcggaaactcatccgatacgtcacagccttcaatggggagctcgaggactatatgagtgaccgggttcagtttgtgatcacagcacaggaatgggatcccagctttgaggaggccctgatggacaacccctccctggcattcgttcgtccccgatggatctacagttgcaatgagaagcagaagttacttcctcaccagctctatggggtggtgccgcaagcctgaagtatgtgctatacacacacacacacacacacacacacacacacacacacgatgcatttaataaagatgagttggttctc

SEQ ID NO: 18 - protein, 633 aa, Homo Sapien
  PEPTIDE (1)...(630): XRCC1
MPEIRLRHVVSCSSQDSTHCAENLLKADTYRKWRAAKAGEKTISVVLQLEKEEQIHSVDIGNDGSAFVEVLVGSSAGGAGEQDYEVLLVTSSFMSPSESRSGSNPNRVRMFGPDKLVRAAAEKRWDRVKIVCSQPYSKDSPFGLSFVRFHSPPDKDEAEAPSQKVTVTKLGQFRVKEEDESANSLRPGALFFSRINKTSPVTASDPAGPSYAAATLQASSAASSASPVSRAIGSTSKPQESPKGKRKLDLNQEEKKTPSKPPAQLSPSVPKRPKLPAPTRTPATAPVPARAQGAVTGKPRGEGTEPRRPRAGPEELGKILQGVVVVLSGFQNPFRSELRDKALELGAKYRPDWTRDSTHLICAFANTPKYSQVLGLGGRIVRKEWVLDCHRMRRRLPSQRYLMAGPGSSSEEDEASHSGGSGDEAPKLPQKQPQTKTKPTQAAGPSSPQKPPTPEETKAASPVLQEDIDIEGVQSEGQDNGAEDSGDTEDELRRVAEQKEHRLPPGQEENGEDPYAGSTDENTDSEEHQEPPDLPVPELPDFFQGKHFFLYGEFPGDERRKLIRYVTAFNGELEDYMSDRVQFVITAQEWDPSFEEALMDNPSLAFVRPRWIYSCNEKQKLLPHQLYGVVPQA

SEQ ID NO: 19 - DNA, 4001 bp, Homo Sapien
  PARP1
aggcatcagcaatctatcagggaacggcggtggccggtgcggcgtgttcggtggcggctctggccgctcaggcgcctgcggctgggtgagcgcacgcgaggcggcgaggcggcagcgtgtttctaggtcgtggcgtcgggcttccggagctttggcggcagctaggggaggatggcggagtcttcggataagctctatcgagtcgagtacgccaagagcgggcgcgcctcttgcaagaaatgcagcgagagcatccccaaggactcgctccggatggccatcatggtgcagtcgcccatgtttgatggaaaagtcccacactggtaccacttctcctgcttctggaaggtgggccactccatccggcaccctgacgttgaggtggatgggttctctgagcttcggtgggatgaccagcagaaagtcaagaagacagcggaagctggaggagtgacaggcaaaggccaggatggaattggtagcaaggcagagaagactctgggtgactttgcagcagagtatgccaagtccaacagaagtacgtgcaaggggtgtatggagaagatagaaaagggccaggtgcgcctgtccaagaagatggtggacccggagaagccacagctaggcatgattgaccgctggtaccatccaggctgctttgtcaagaacagggaggagctgggtttccggcccgagtacagtgcgagtcagctcaagggcttcagcctccttgctacagaggataaagaagccctgaagaagcagctcccaggagtcaagagtgaaggaaagagaaaaggcgatgaggtggatggagtggatgaagtggcgaagaagaaatctaaaaaagaaaaagacaaggatagtaagcttgaaaaagccctaaaggctcagaacgacctgatctggaacatcaaggacgagctaaagaaagtgtgttcaactaatgacctgaaggagctactcatcttcaacaagcagcaagtgccttctggggagtcggcgatcttggaccgagtagctgatggcatggtgttcggtgccctccttccctgcgaggaatgctcgggtcagctggtcttcaagagcgatgcctattactgcactggggacgtcactgcctggaccaagtgtatggtcaagacacagacacccaaccggaaggagtgggtaaccccaaaggaattccgagaaatctcttacctcaagaaattgaaggttaaaaaacaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcatcagcaccaaaaaggaggtggaaaagatgaataagaagatggaggaagtaaaggaagccaacatccgagttgtgtctgaggacttcctccaggacgtctccgcctccaccaagagccttcaggagttgttcttagcgcacatcttgtccccttggggggcagaggtgaaggcagagcctgttgaagttgtggccccaagagggaagtcaggggctgcgctctccaaaaaaagcaagggccaggtcaaggaggaaggtatcaacaaatctgaaaagagaatgaaattaactcttaaaggaggagcagctgtggatcctgattctggactggaacactctgcgcatgtcctggagaaaggtgggaaggtcttcagtgccacccttggcctggtggacatcgttaaaggaaccaactcctactacaagctgcagcttctggaggacgacaaggaaaacaggtattggatattcaggtcctggggccgtgtgggtacggtgatcggtagcaacaaactggaacagatgccgtccaaggaggatgccattgagcacttcatgaaattatatgaagaaaaaaccgggaacgcttggcactccaaaaatttcacgaagtatcccaaaaagttctaccccctggagattgactatggccaggatgaagaggcagtgaagaagctgacagtaaatcctggcaccaagtccaagctccccaagccagttcaggacctcatcaagatgatctttgatgtggaaagtatgaagaaagccatggtggagtatgagatcgaccttcagaagatgcccttggggaagctgagcaaaaggcagatccaggccgcatactccatcctcagtgaggtccagcaggcggtgtctcagggcagcagcgactctcagatcctggatctctcaaatcgcttttacaccctgatcccccacgactttgggatgaagaagcctccgctcctgaacaatgcagacagtgtgcaggccaaggtggaaatgcttgacaacctgctggacatcgaggtggcctacagtctgctcaggggagggtctgatgatagcagcaaggatcccatcgatgtcaactatgagaagctcaaaactgacattaaggtggttgacagagattctgaagaagccgagatcatcaggaagtatgttaagaacactcatgcaaccacacacaatgcgtatgacttggaagtcatcgatatctttaagatagagcgtgaaggcgaatgccagcgttacaagccctttaagcagcttcataaccgaagattgctgtggcacgggtccaggaccaccaactttgctgggatcctgtcccagggtcttcggatagccccgcctgaagcgcccgtgacaggctacatgtttggtaaagggatctatttcgctgacatggtctccaagagtgccaactactgccatacgtctcagggagacccaataggcttaatcctgttgggagaagttgcccttggaaacatgtatgaactgaagcacgcttcacatatcagcaagttacccaagggcaagcacagtgtcaaaggtttgggcaaaactacccctgatccttcagctaacattagtctggatggtgtagacgttcctcttgggaccgggatttcatctggtgtgaatgacacctctctactatataacgagtacattgtctatgatattgctcaggtaaatctgaagtatctgctgaaactgaaattcaattttaagacctccctgtggtaattgggagaggtagccgagtcacacccggtggctctggtatgaattcacccgaagcgcttctgcaccaactcacctggccgctaagttgctgatgggtagtacctgtactaaaccacctcagaaaggattttacagaaacgtgttaaaggttttctctaacttctcaagtcccttgttttgtgttgtgtctgtggggaggggttgttttggggttgtttttgttttttcttgccaggtagataaaactgacatagagaaaaggctggagagagattctgttgcatagactagtcctatggaaaaaaccaagcttcgttagaatgtctgccttactggtttccccagggaaggaaaaatacacttccacccttttttctaagtgttcgtctttagttttgattttggaaagatgttaagcatttatttttagttaaaaataaaaactaatttcatactatttagattttcttttttatcttgcacttattgtcccctttttagttttttttgtttgcctcttgtggtgaggggtgtgggaagaccaaaggaaggaacgctaacaatttctcatacttagaaacaaaaagagctttccttctccaggaatactgaacatgggagctcttgaaatatgtagtattaaaagttgcatttgaaattcttgactttcttatgggcacttttgtcttccaaattaaaactctaccacaaatatacttacccaagggctaatagtaatactcgattaaaaatgcagatgccttctctaaaaaaaaaaaaaaaaa

SEQ ID NO: 20 - protein, 1014 aa, Homo Sapien
  PEPTIDE (1)...(1010): PARP1
MAESSDKLYRVEYAKSGRASCKKCSESIPKDSLRMAIMVQSPMFDGKVPHWYHFSCFWKVGHSIRHPDVEVDGFSELRWDDQQKVKKTAEAGGVTGKGQDGIGSKAEKTLGDFAAEYAKSNRSTCKGCMEKIEKGQVRLSKKMVDPEKPQLGMIDRWYHPGCFVKNREELGFRPEYSASQLKGFSLLATEDKEALKKQLPGVKSEGKRKGDEVDGVDEVAKKKSKKEKDKDSKLEKALKAQNDLIWNIKDELKKVCSTNDLKELLIFNKQQVPSGESAILDRVADGMVFGALLPCEECSGQLVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEHFMKLYEEKTGNAWHSKNFTKYPKKFYPLEIDYGQDEEAVKKLTVNPGTKSKLPKPVQDLIKMIFDVESMKKAMVEYEIDLQKMPLGKLSKRQIQAAYSILSEVQQAVSQGSSDSQILDLSNRFYTLIPHDFGMKKPPLLNNADSVQAKVEMLDNLLDIEVAYSLLRGGSDDSSKDPIDVNYEKLKTDIKVVDRDSEEAEIIRKYVKNTHATTHNAYDLEVIDIFKIEREGECQRYKPFKQLHNRRLLWHGSRTTNFAGILSQGLRIAPPEAPVTGYMFGKGIYFADMVSKSANYCHTSQGDPIGLILLGEVALGNMYELKHASHISKLPKGKHSVKGLGKTTPDPSANISLDGVDVPLGTGISSGVNDTSLLYNEYIVYDIAQVNLKYLLKLKFNFKTSLW

SEQ ID NO: 21 - DNA, 601 bp, Homo Sapien
  XPD
ccagtgaactgcgctggccgcagcttcttgggaacagtgcaggagggatgggctggtggggtgagagggggtctatcatctcctggcccccccttgcstctgggtacctggtggatagctgccttctcctgcgattaaaggctgtggacntgacagtgagaaatgtcacctgacttcataagaccttctagcaccaccrccgctgggaaccagggccaggcaagactcaggagtcaccaggaaccgtttatggccccacccgccccactcagagctgctgagcaatctgctctatcctctacagcgtctcctctgattctagctgctccaggctgagcagggacaggcccagctgatcctcctgcagagaacagaggaaagggagaggggggcactgttgggcaggggcccaggcagctgctgcacttccccaaccaccccscngaatggccagtagggacaggatgtttgaatgaatttggagatgtccgtataatccagagcgggccatccctgtcaacataagatggggctgagggaggagccagctaggragtmggagaaggaccggctttctccaggccagcaccgtctctcctga

SEQ ID NO: 22 - DNA, 601 bp, Homo Sapien
  PARP1
gacccatttgtctaaagtaccttcactgtaggtcccaaatgtcagcatgtacgagaggaaagacagttcttccacctctcaactcccccaggccacaaggccttgttaacattccagaaagtccttatgagctctcaatatgtgcagcaggtaacaggctggccctgaccagcaggagggtttgccattcactgtgttggaccttctctgcatgtaggttttctctgccacctgggtgagtctgtctcattcaccatgatacctaagtcgggggctttcttttgctcctccaggccaaggyggaaatgcttgacaacctgctggacatcgaggtggcctacagtctgctcaggggagggtctgatgatagcagcaaggatcccatcgatgtcaactatgagaagctcaaaactgacattaaggtaacagggtcaggccttcctcaagcatcttaacctctcatcctgagagcagcggtcccaaacttccatgtgtgttaggatctcctggaaagcttgttaaaacacaggtggctggcgcccaataattcacttttctaacaagttcccggacattgctgctggtggtcctttgagaacct